(19) United States
(12) Patent Application Publication
(10) Pub. No.: US 2023/0075875 A1
(43) Pub. Date: Mar. 9, 2023

(54) METHOD AND TRANSCATHETER ANNULOPLASTY SYSTEM FOR CONTRACTING HEART TISSUE

(71) Applicant: HANGZHOU VALGEN MEDTECH CO., LTD., Hangzhou (CN)

(72) Inventors: Tingchao ZHANG, Hangzhou (CN); Ronghui GUO, Hangzhou (CN); Jingxu KUANG, Hangzhou (CN)

(21) Appl. No.: 17/900,023

(22) Filed: Aug. 31, 2022

(30) Foreign Application Priority Data
Sep. 3, 2021 (CN) 202111031389.2
Sep. 3, 2021 (CN) 202111032580.9
Dec. 31, 2021 (CN) 202111677720.8
Dec. 31, 2021 (CN) 202111677919.0

(51) Int. Cl. A61F 2/24

(52) U.S. Cl. CPC A61F 2/2466; A61F 2/2445; A61F 2220/0016; A61F 2220/0075; A61F 2250/0012; A61F 2230/0013

(57) ABSTRACT

The present disclosure provides a method for contracting heart tissue using a transcatheter annuloplasty system. The system includes a plurality of anchoring components, a flexible tightening string and a flexible delivery member. The tightening string is coupled with a first anchoring component and the delivery member. The method includes: implanting the first anchoring component into the heart tissue, the tightening string entering a body along with the first anchoring component, and a proximal end of the delivery member extending outside the body; delivering each of remaining anchoring components, along the delivery member, to the tightening string and implanting it into the heart tissue; and tightening the tightening string. Therefore, after the plurality of anchoring components are implanted at different positions, along a circumferential direction of the mitral annulus, the tightening string is tightened to adjust a spacing of the anchoring components so as to contract the heart tissue.

### CROSS-REFERENCE TO RELATED APPLICATIONS

This application claims priority to China Patent Application No. 202111032580.9, filed on Sep. 3, 2021, and China Patent Application No. 202111031389.2, filed on Sep. 3, 2021, and China Patent Application No. 202111677720.8, filed on Dec. 31, 2021, and China Patent Application No. 202111677919.0, filed on Dec. 31, 2021, all of which are hereby incorporated in their entirety by reference.

### TECHNICAL FIELD

The present disclosure relates to the technical fields of medical devices, and in particular, to a method and transcatheter annuloplasty system for contracting heart tissue.

### BACKGROUND

With the aging of the population, the incidence of valvular disease has gradually increased, and the incidence of mitral regurgitation (MR) has also shown a significant upward trend. Mitral regurgitation includes primary mitral regurgitation and secondary mitral regurgitation. The primary mitral regurgitation results in poor anastomosis of the anterior and posterior mitral leaflets due to abnormal mitral valve leaflets, rupture of the chordae tendineae, or papillary muscle insufficiency. The secondary mitral regurgitation results in poor anastomosis of the anterior and posterior mitral leaflets due to annulus dilatation, left atrium and left ventricle enlargement. In recent years, mitral valve interventional therapy has developed rapidly, mainly including valve repair or valve replacement. Mitral valve annuloplasty is a common repair procedure that eliminates or reduces mitral regurgitation by reducing a size of the mitral annulus of patient.

In the prior art, a plurality of anchors slidably coupled to a tether are sequentially implanted in a mitral annulus through a transcatheter route, and then the tether is tightened to contract the mitral annulus, thereby improving mitral regurgitation. Since the anchor needs to be threaded on the tether from outside the body, the tether extends from the mitral annulus to outside the body, and the plurality of anchors are implanted in the mitral annulus, the tether is pulled to achieve an effect of improving mitral regurgitation, the tether needs to be locked so that the tightened tether is locked on the mitral annulus to maintain a certain length, and then the excess tether is trimmed off. However, cutting the tether in the body can cause material particles to dislodge, potentially causing embolism.

### SUMMARY

In a first aspect, the present disclosure provides a method for contracting heart tissue using a transcatheter annuloplasty system. The transcatheter annuloplasty system includes a plurality of anchoring components, a tightening string, and a delivery member. Both the tightening string and the delivery member are flexible. A distal end of the tightening string is coupled to a first of the plurality of anchoring components. A distal end of the delivery member is coupled to a proximal end of the tightening string. The method includes: implanting the first of plurality of anchoring components into the heart tissue, the tightening string entering a body of a patient along with the first of the plurality of anchoring components, and a proximal end of the delivery member extending outside the body; and delivering each of remaining of the plurality of anchoring components, along the delivery member, to the tightening string and implanting each of remaining of the plurality of anchoring components into the heart tissue in sequence; and tightening the tightening string thereby adjusting a spacing of the plurality of anchoring components to contract a size of the heart tissue.

In the method for contracting heart tissue using the transcatheter annuloplasty system provided in the present disclosure, the plurality of anchoring components are delivered, along the delivery member, to the tightening string; after the plurality of anchoring components are implanted into the heart tissue, the tightening string is tightened to adjust the spacing of the plurality of anchoring components, which can achieve a purpose of contracting the size of the heart tissue. In this way, an appropriate implanting length of the tightening string can be selected, so that the tightening string does not need to be cut in the body, the shedding of the string particles caused by the tightening string can be avoided, and the operation is safer.

In a second aspect, the present disclosure provides a method for contracting heart tissue using a transcatheter annuloplasty system. The transcatheter annuloplasty system includes a plurality of anchoring components, a tightening string, and a delivery sheath. The tightening string is flexible. A distal end of the tightening string is coupled to a first of the plurality of anchoring components. A tube wall of the delivery sheath defines a through slot extending from a distal end of the delivery sheath towards a proximal end of the delivery sheath. A part of the anchoring component is exposed from the through slot. The method includes: implanting the first of the plurality of anchoring components into the heart tissue; delivering each of remaining of the plurality of anchoring components to the tightening string and implanting each of remaining of the plurality of anchoring components into the heart tissue in sequence; tightening the tightening string, thereby adjusting a spacing of the plurality of anchoring components to contract a size of the heart tissue.

In the method for contracting heart tissue using the transcatheter annuloplasty system provided in the present disclosure, the plurality of anchoring components are delivered to the heart tissue through the delivery sheath; after the plurality of anchoring components are implanted into the heart tissue, the tightening string is tightened to contract the spacing of the plurality of anchoring components, which can achieve a purpose of contracting the size of the heart tissue. A part of the anchoring component exposes from the through slot of the delivery sheath; the tightening string can be thus coupled to the part of the anchoring component exposed from the delivery sheath; and a part of the tightening string extending to the distal end from where it couples the anchoring component is located outside the delivery sheath, which can prevent the tightening string from being entangled with a part of the anchoring component in the lumen of the delivery sheath, and is beneficial to the implantation of the anchoring component successfully.

### BRIEF DESCRIPTION OF THE ACCOMPANYING DRAWINGS

In order to illustrate the technical solutions of the embodiments of the present disclosure more clearly, the following briefly introduces the accompanying drawings that need to be used in the embodiments. As far as technical personnel are concerned, other drawings can also be obtained based on these drawings without any creative effort.

FIG. 1 illustrates a schematic structural diagram of a transcatheter annuloplasty system, according to one embodiment of the present disclosure.

FIG. 2 illustrates a schematic diagram of the implants being implanted into a mitral annulus and the tightening string being not tightened, according to one embodiment of the present disclosure.

FIG. 3 illustrates a schematic diagram of the tightening string, of FIG. 2, being tightened.

FIG. 4 illustrates a schematic diagram of a coupling between a delivery member, a tightening string and a first of the plurality of anchoring components, according to one embodiment of the present disclosure.

FIG. 5 illustrates a schematic diagram of a coupling between a delivery member, a tightening string and a first of the plurality of anchoring components, according to another embodiment of the present disclosure.

FIG. 6 illustrates a schematic diagram of a coupling between a delivery member, a tightening string and a first of the plurality of anchoring components, according to another embodiment of the present disclosure.

FIG. 7 illustrates a schematic diagram of a coupling between the tightening string and a threading structure of the first of the plurality of anchoring components, according to one embodiment of the present disclosure.

FIG. 8 illustrates a schematic structural diagram of an anchoring component, according to one embodiment of the present disclosure.

FIG. 9 illustrates a schematic structural diagram of an anchoring component, according to another embodiment of the present disclosure.

FIG. 10 illustrates a schematic structural diagram of an anchoring component, according to another embodiment of the present disclosure.

FIG. 11 illustrates a schematic diagram of a coupling between an anchoring component and an anchoring apparatus, according to one embodiment of the present disclosure.

FIG. 12 illustrates a schematic diagram of a separation of an anchoring component and an anchoring apparatus shown in FIG. 11.

FIG. 13 illustrates an axial cross-sectional view of an anchoring component of FIG. 11 when coupled to the anchoring apparatus.

FIG. 14 illustrates a schematic structural diagram of an anchoring component being inserted in a delivery sheath, according to one embodiment of the present disclosure.

FIG. 15 illustrates an enlarged schematic view of a part XV shown in FIG. 14.

FIG. 16 illustrates a schematic structural diagram of an anchoring component being inserted in a delivery sheath, according to another embodiment of the present disclosure.

FIG. 17 illustrates an enlarged schematic view of a part XVII shown in FIG. 16.

FIG. 18 illustrates an exploded schematic structural diagram of a delivery sheath, an anchoring component, and a delivery member, according to one embodiment of the present disclosure.

FIG. 19 illustrates a schematic diagram of a push member pushing a spacer, according to one embodiment of the present disclosure.

FIG. 20 illustrates a schematic structural diagram of a guide apparatus, a delivery sheath, and an anchoring apparatus, according to one embodiment of the present disclosure.

FIG. 21 illustrates a perspective view of a delivery sheath pushing a spacer in a delivery channel of a guide apparatus, according to one embodiment of the present disclosure.

FIG. 22 illustrates a schematic structural diagram of a retractor and a delivery member, according to one embodiment of the present disclosure.

FIG. 23 illustrates a partial perspective structural diagram of the retractor shown in FIG. 22.

FIG. 24 illustrates another partial perspective structural diagram of the retractor shown in FIG. 22.

FIG. 25 illustrates an axial cross-sectional view of a coupling between the retractor and a distal end of an adjusting apparatus, according to one embodiment of the present disclosure.

FIG. 26 illustrates a flowchart of a method for contracting heart tissue using a transcatheter annuloplasty system, according to one embodiment of the present disclosure.

FIG. 27 illustrates another flowchart of a method for contracting heart tissue using a transcatheter annuloplasty system, according to one embodiment of the present disclosure.

FIG. 28 illustrates another flowchart of a method for contracting heart tissue using a transcatheter annuloplasty system, according to one embodiment of the present disclosure.

FIG. 29 illustrates another flowchart of a method for contracting heart tissue using a transcatheter annuloplasty system, according to one embodiment of the present disclosure.

FIG. 30 illustrates a flowchart of a method for contracting heart tissue using a transcatheter annuloplasty system, according to another embodiment of the present disclosure.

FIG. 31 illustrates a flowchart of a method for contracting mitral annulus using a transcatheter annuloplasty system, according to one embodiment of the present disclosure.

FIGS. 32 to 40 illustrate schematic diagrams of a using process of a transcatheter annuloplasty system, according to one embodiment of the present disclosure.

### DETAILED DESCRIPTION OF ILLUSTRATED EMBODIMENTS

The technical solutions in the embodiments of the present disclosure will be clearly and completely described below with reference to the accompanying drawings in the embodiments of the present disclosure. Obviously, the described embodiments are only a part of the embodiments of the present disclosure, but not all of the embodiments. Based on the embodiments in the present disclosure, all other embodiments obtained by those of ordinary skill in the art without creative work fall within a protection scope of the present disclosure.

Furthermore, the following descriptions of the various embodiments are configured to illustrate specific embodiments in which the present disclosure may be practiced with reference to the accompanying drawings. Directional terms mentioned in the present disclosure, such as “upper”, “lower”, “front”, “rear”, “left”, “right”, “inner”, “outer”, “side”, and the like are directions only referred to the accompanying drawings. Therefore, the directional terms used are for better and clearer description and understanding of the present disclosure, rather than indicating or implying that the referred device or element must have a specific orientation, in a specific orientation construction and operation, and therefore should not be construed as limitations on the present disclosure.

It should be noted that, in order to more clearly describe a structure of a transcatheter annuloplasty system provided by the present disclosure, the definitions terms “proximal” and “distal” described in the specification of the present disclosure are conventional terms in the field of interventional medicine. Specifically, “distal end” refers to an end away from an operator during a surgical operation, and “proximal end” refers to an end close to an operator during a surgical operation; a direction of a central axis of rotation of objects such as cylinders and tubes is defined as an axial direction; a circumferential direction is a direction around an axis of objects such as cylinders, tubes, and the like (perpendicular to the axis and perpendicular to a radius of a cross-section); a radial direction is a direction along an diameter or a radius. It is worth noting that regardless of “proximal end”, “distal end”, “one end”, “another end”, “first end”, “second end”, “initial end”, “tail end”, “both ends”, “free end”, “upper end”, “lower end” and other words appearing in the “end” is not limited to end point, or end surface, but also includes the part that extends an axial distance and/or a radial distance from the end point or end surface on the element to which the end point or end surface belongs. Unless otherwise defined, all technical and scientific terms used in the present disclosure have the same meaning as commonly understood by one of ordinary skill in the technical field to which the present disclosure belongs. The common terms used in the specification of the present disclosure are only for the purpose of describing specific embodiments, and should not be construed as limitations of the present disclosure.

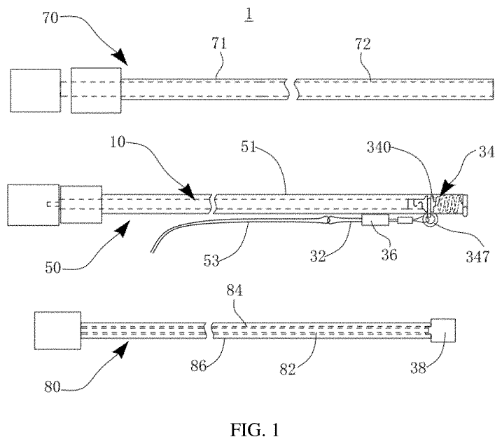

Referring to FIGS. 1 to 3, the present disclosure provides a transcatheter annuloplasty system 1. The transcatheter annuloplasty system 1 may include an anchoring apparatus 10, an implant 30, a delivery apparatus 50 and a guide apparatus 70.

The transcatheter annuloplasty system 1 can be configured to implant the implant 30 into a heart tissue such as a mitral annulus or a tricuspid annulus. The implant 30 may include a tightening string 32 and a plurality of anchoring components 34 coupled by the tightening string 32 in series. The spacing between the plurality of anchoring components 34 can be contracted by tightening the tightening string 32, so as to directly contract the mitral annulus, thereby achieving a treatment of mitral regurgitation or tricuspid regurgitation. Of course, the transcatheter annuloplasty system 1 can also be configured to implant a plurality of anchoring components 34, series coupled by tightening string 32, into heart tissues such as the left ventricular wall under the mitral annulus or the right ventricular wall under the tricuspid annulus. The spacing of the plurality of anchoring components 34 can be contracted by tightening the tightening string 32, so as to contract the ventricle, thereby reducing a volume of the ventricle, to achieve the same purpose of annuloplasty. This sub-annular shaping can preserve a natural structure of the mitral valve and tricuspid valve. The implant 30 implanted in the left ventricular wall is particularly suitable for treating heart failure and functional mitral regurgitation caused by abnormal left ventricular function. Hereinafter, a detailed description will be given by taking a heart tissue as the mitral annulus as an example.

Referring to FIG. 1, the guide apparatus 70 has a channel. In some embodiments, the guide apparatus 70 includes a first guide sheath 71 and a second guide sheath 72 inserted into the first guide sheath 71. The second guide sheath 72 can extend out from a distal end of the first guide sheath 71 and be adjusted to a vicinity of the mitral annulus, thereby establishing an intervention channel from outside the body to the heart, that is, establishing a delivery channel from outside the body to the heart tissue.

Preferably, both the first guide sheath 71 and the second guide sheath 72 are steerable sheaths, which are convenient to adjust a bending angle and direction at a distal end of the guide apparatus 70. The two steerable guide sheaths can better adjust the bending angle and direction at the distal end of the guide apparatus 70. Of course, in other embodiments, the guide apparatus 70 may employ only one steerable guide sheath. The steerable guide sheath is a commonly used guide apparatus in an interventional surgery in a prior art, and the details are not described here.

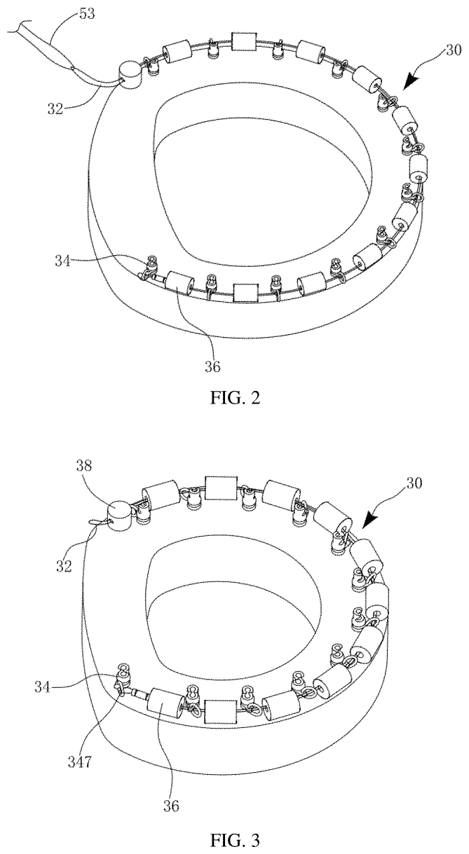

Referring to FIGS. 2 and 3, in some embodiments, the implant 30 includes a tightening string 32 having flexibility and a plurality of anchoring components 34; the plurality of anchoring components 34 are coupled by the tightening string 32 and are respectively configured to implant into the mitral annulus.

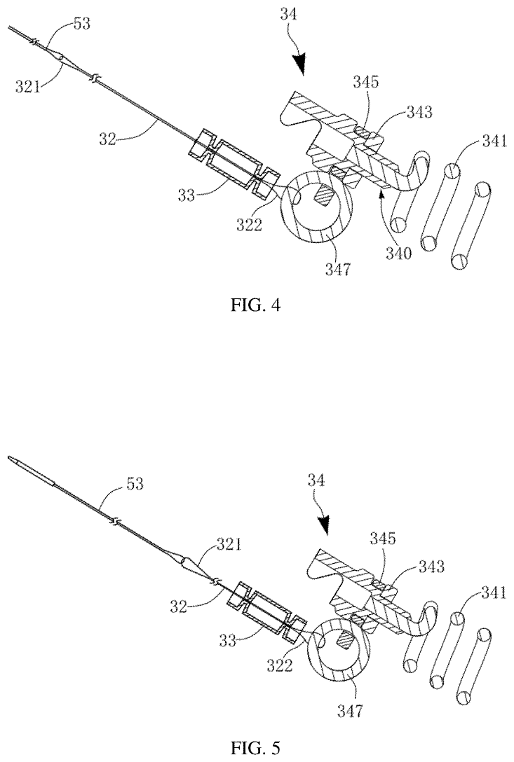

As shown in FIG. 1, each anchoring component 34 is detachably coupled to a distal end of the anchoring apparatus 10. The anchoring apparatus 10 is configured to drive the anchoring component 34 to be implanted into the mitral annulus. The delivery apparatus 50 includes a delivery sheath 51 and a flexible delivery member 53. A distal end of the delivery member 53 is coupled to a proximal end of the tightening string 32. A proximal end of the delivery member 53 extends outside the body of the patient. A distal end of the tightening string 32 is coupled to a first of the plurality of anchoring components 34 (hereinafter “first anchoring component 34”) for being implanted into the mitral annulus (see FIGS. 4 to 7). Remaining of the plurality of anchoring components 34 (hereinafter “remaining anchoring components 34”) for being implanted into the mitral annulus can be delivered, along the delivery member, 53 and threaded onto the tightening string 32. After the plurality of anchoring components 34 are implanted into the mitral annulus, the tightening string 32 is tightened to adjust the spacing of the plurality of anchoring components 34. In this way, the plurality of anchoring components 34 are coupled in series through the tightening string 32 and implanted at different positions along a circumference of the mitral annulus, and the tightening string 32 is tightened to contract the spacing of the plurality of anchoring components 34, thereby achieving a purpose of contracting the mitral annulus.

It can be understood that, in the transcatheter annuloplasty system 1 provided by the present disclosure, the delivery member 53 detachably coupled to the tightening string 32 is provided, and the plurality of anchoring components 34 are sequentially threaded onto the tightening string 32 through the delivery member 53, so that the tightening string 32 can be selected with an appropriate implanting length, the tightening string 32 does not need to be cut in the body, and the shedding of the string particles caused by the tightening string 32 can be avoided, and the operation is safer and more simple.

It should be noted that the tightening string 32 has a certain axial length and flexibility, and the radial cross-sectional shape of the tightening string 32 can be circular, oblate, rectangular, square, or other shapes. Similarly, the delivery member 53 also has a certain axial length and flexible, the radial cross-sectional shape of the delivery member 53 may also be circular, oblate, rectangular, square or other shapes. the present disclosure does not specifically limit the radial cross-sectional shape of the tightening string 32 and the delivery member 53.

Please referring to FIG. 1, the anchoring apparatus 10 is inserted into the lumen of the delivery sheath 51, the anchoring components 34 are detachably coupled to a distal end of the anchoring apparatus 10 and inserted into the delivery sheath 51. The delivery sheath 51 is configured to deliver the anchoring components 34 to the mitral annulus. A distal part of the delivery sheath 51 is flexible. The delivery sheath 51 is inserted into an lumen of the second guide sheath 72, and a distal end of the delivery sheath 51 can be driven to bend by adjusting a bending angle of a distal end of the guide apparatus 70.

When using the transcatheter annuloplasty system 1 for contracting the mitral annulus, an interventional channel from outside the body of the patient to the heart of the patient is first established through the guide apparatus 70; then the delivery sheath 51 equipped with the anchoring apparatus 10 and the anchoring component 34, together with the tightening string 32 and the delivery member 53 are moved axially towards the distal end in a lumen of the second guide sheath 72 of the guide apparatus 70 until the delivery sheath 51 abuts the mitral annulus; and then the anchoring component 34 is driven by the anchoring apparatus 10 within the delivery sheath 51 to be implanted into the mitral annulus. The plurality of anchoring components 34 are respectively implanted into different positions, along a circumferential direction of the mitral annulus, through same steps as described above (as shown in FIG. 2), the spacing of the plurality of anchoring components 34 can be adjusted by tightening the tightening string 32, which causes the plurality of anchoring components 34 to be relatively gathered (as shown in FIG. 3), thereby driving the mitral annulus to contract, so as to achieve a purpose of contracting the mitral annulus.

Referring to FIG. 4, in some embodiments, each anchoring component 34 includes an anchoring member 340 and a threading structure. The anchoring member 340 is configured to anchor into the mitral annulus. The threading structure is configured to couple the tightening string 32. The distal end of the tightening string 32 is coupled to the threading structure of the first anchoring component 34 for implanting into the mitral annulus. The proximal end of the tightening string 32 slides through the threading structure of the other anchoring components 34 for implanting into the mitral annulus. In this way, the tightening string 32 is coupled to the anchoring member 340 through the threading structure, thereby coupling the plurality of anchoring components 34 implanted into the mitral annulus in series, and tightening the tightening string 32 can contract the spacing of the plurality of anchoring components 34, thereby achieving a purpose of contracting the mitral annulus.

Referring to FIGS. 4 to 6, in some embodiments, the proximal end of the tightening string 32 forms a first loop 321; after passing through the first loop 321, the delivery member 53 is folded in half so that the distal end of the delivery member 53 is coupled to the tightening string 32 in a U-shaped, that is detachable coupling. In this embodiment, the delivery member 53 can be an elongated string including two head ends. After the delivery member 53 is coupled to the tightening string 32, the delivery member 53 can be a double-string structure in a U-shape. When the two head ends of the delivery member 53 are not closed, the delivery member 53 can be directly pulled out from the first loop 321 to separate the delivery member 53 from the tightening string 32 and withdrawn to outside the body of patient, without the need for cutting the delivery member 53. When the two head ends of the delivery member 53 are closed, the delivery member 53 can be cut outside the body so that the delivery member 53 has a non-closed double-string structure, and the delivery member 53 can be withdrawn.

In the example of FIGS. 4 and 5, the tightening string 32 is a double-string structure. The tightening string 32 can be an elongated string including two head ends. One head end of the tightening string 32 passes through the threading structure of the first anchoring component 34 for implanting into the mitral annulus to form a U-shaped connection. The two head ends of the tightening string 32 are fixed through a pressing tube 33 so that the tightening string 32 is in a closed double-string structure. The tightening string 32 forms a first loop 321 at a proximal end and a second loop 322 at a distal end on both sides of the pressing tube 33, respectively. The distal end of the delivery member 53 and the first loop 321 at the proximal end of the tightening string 32 form a U-shaped connection. The threading structure of the first anchoring component 34 and the second loop 322 at the distal end of the tightening string 32 form a U-shaped connection.

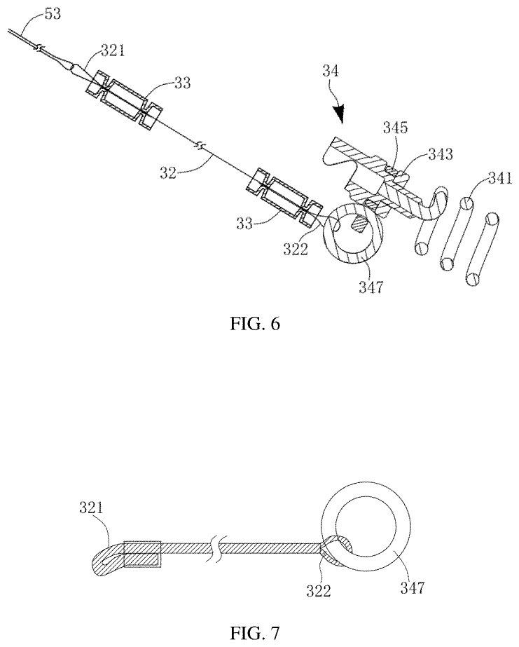

In the example of FIG. 6, the tightening string 32 is a single-string structure. The tightening string 32 can be an elongated string including two head ends. One head end of the tightening string 32 passes through the threading structure of the first anchoring component 34 for implanting into the mitral annulus to form a U-shaped connection, and is fixed by one pressing tube 33 to make the distal end of the tightening string 32 form the second loop 322. Another head end of the tightening string 32 forms the first loop 321 at its proximal end and is fixed by another pressing tube 33. The first loop 321 forms a U-shaped connection with the distal end of the delivery member 53, and the second loop 322 is configured to form a U-shaped connection with the threading structure of the first anchoring component 34. It can be understood that the tightening string 32 having the single-string structure reduce a risk of winding and crimping between the tightening string 32 and the anchoring components 34, and the operation is safer.

It should be noted that the pressing tube 33 may be replaced by other fixing parts such as a clamp. Furthermore, the two head ends of the tightening string 32 may also form the first loop 321 and the second loop 322 by bonding or knotting, which is not limited. The tightening string 32 may be made of a single strand wire or braided with a plurality of wires, preferably, the tightening string 32 is not easily broken.

Referring to FIG. 7, in some embodiments, the tightening string 32 is a metal wire tube with good flexibility, and the metal wire tube is woven from a plurality of metal wires. The metal wire may be but not limited to nickel-titanium wire, tungsten wire, and the like. The metal wire tube is coupled to the threading structure of the first anchoring component 34 for implanting into the mitral annulus during braiding, so that the distal end of the metal wire tube is formed with the second loop 322, and the proximal end of the metal wire tube is bent to form the first loop 321, and is also fixed with the pressing tube 33. It can be understood that by using the metal wire tube as the tightening string 32, only one pressing tube 33 can form a single-string structure with the first loop 321 and the second loop 322, reducing the number of pressing tubes 33. The proximal end of the metal wire tube may also be braided to form the first loop 321.

In some embodiments, a stiffness of the tightening string 32 is greater than or equal to a stiffness of the delivery member 53, preferably greater than the stiffness of the delivery member 53. In this way, the tightening string 32 has a high hardness and is not easy to break. After the implant 30 is implanted into the mitral annulus, the tightening string 32 can stably couple the plurality of anchoring components 34 in series, so as to avoid the tightening string 32 from being broken due to the long-term action of the valve leaflet.

In order to ensure the safety of implantation, both the tightening string 32 and the delivery member 53 can be made of biocompatible metal materials or polymer materials, especially the tightening string 32. The metal materials include but are not limited to at least one of tungsten, nickel-titanium, tantalum, gold, stainless steel and alloys thereof which have a function of developing. The polymer material includes but not limited to at least one of polyethylene, polyamide, polypropylene, polyurethane and ultra-high molecular weight polyethylene. Specifically, in some embodiments, the tightening string 32 is preferably made of a metal material with good biocompatibility, and the delivery member 53 is made of a biocompatible polymer material. Since the metal material used in the tightening string32 is radiopaque, during the implantation of all anchoring components 34, the state and position of the tightening string 32 can be seen through a digital subtraction angiography equipment or computed tomography equipment, which is beneficial to reduce a risk of winding during the operation and improve a success rate of the operation.

Please referring to FIG. 1 and FIGS. 8 to 10 together, in some embodiments, the anchoring apparatus 10 drives the anchoring member 340 to rotate to anchor into the heart tissue; the threading structure can be rotatably disposed on the anchoring member 340. In this way, during a rotation of the anchoring member 340, since the anchoring member 340 and the threading structure can rotate relative to each other, the threading structure will not follow the rotation of the anchoring member 340, and the tightening string 32 coupled to the threading structure is prevented from being entangled with the anchoring component 34. Specifically, the threading structure includes a connecting member 345 and a threading ring 347. The connecting member 345 is movably sleeved on the anchoring member 340, the threading ring 347 is movably coupled with the connecting member 345, and the threading ring 347 is configured to couple the tightening string 32.

It can be understood that, in the anchoring components 34 provided in the present disclosure, since the connecting member 345 can be rotated 360° around a central axis of the anchoring member 340, and the threading ring 347 is movably coupled with the connecting member 345, after the plurality of anchoring components 34 are implanted into the mitral annulus, when the tightening string 32 is tightened, under the pulling force of the tightening string 32, the threading ring 347 of the anchoring components 34 can move to a state after the tightening string 32 is tightened. The threading ring 347 of each anchoring component 34 can be moved so that the threading direction is along the circumferential direction of the mitral annulus, thereby greatly reducing the resistance of the tightening string 32 during the contraction process, and the tightening string 32 is not bent, and the contraction is stable and smooth, so as to ensure the stability of annuloplasty, and the annuloplasty effect is good. Furthermore, since the resistance of the tightening string 32 in the tightening direction is greatly reduced, the tightening force is reduced and the tightening force is distributed more evenly on each anchoring component 34, so that the tightening force on each anchoring component 34 is greatly reduced, reducing the force of each anchoring component 34 on the mitral annulus, reducing the risk of valve damage, avoiding the situation where a single anchoring component 34 is subject to a large part of the tightening force, and reducing a risk of falling off of the anchoring component 34, therefore, the implantation is safer.

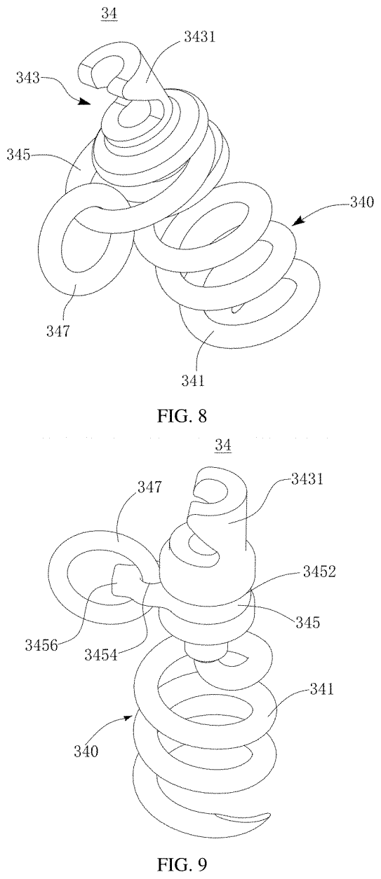

Referring to FIGS. 8 to 10, the anchoring member 340 includes an anchoring part 341 and an anchoring base 343 coupled to a proximal end of the anchoring part 341. The anchoring part 341 is configured to anchor into heart tissue such as the mitral annulus. The anchoring base 343 is configured to detachably couple the anchoring apparatus 10. An outer wall of the anchoring base 343 defines a connecting groove along a circumferential direction thereof, and the connecting member 345 is movably sleeved on the anchoring base 343 and partially received in the connecting groove.

In other embodiments, the connecting member 345 can also be movably sleeved on a proximal end of the anchoring part 341, and the anchoring part 341 can be a helical anchor or has a limiting member so that the connecting member 345 does not drop from the anchoring member 340. Of course, the threading structure of the anchoring component 34 may also only include a threading ring sleeved on the anchoring member 340, preferably, the threading ring can be movably sleeved on the anchoring base 343. The threading structure can also be a threading eyelet defined on the anchoring member 340, and preferably, the threading eyelet can be defined on the anchoring base 343.

In the illustrated example, the anchoring part 341 of the anchoring component 34 is a helical anchor, and the helical anchor has a tip, which is easy to pierce into the mitral annulus or other tissues, and the implantation is stable. Although the anchoring part 341 is illustrated herein as a helical anchor, in other embodiments, the anchoring part 341 may be another suitable type having a structure that enables the anchoring part 341 to engage and substantially secure to heart tissue, such as but not limited to barbs, hooks, tines, and the like.

In the example of FIG. 8, the connecting member 345 is a connecting ring movably sleeved on the anchoring base 343, the connecting ring is partially received in the connecting groove and is directly coupled with the threading ring 347.

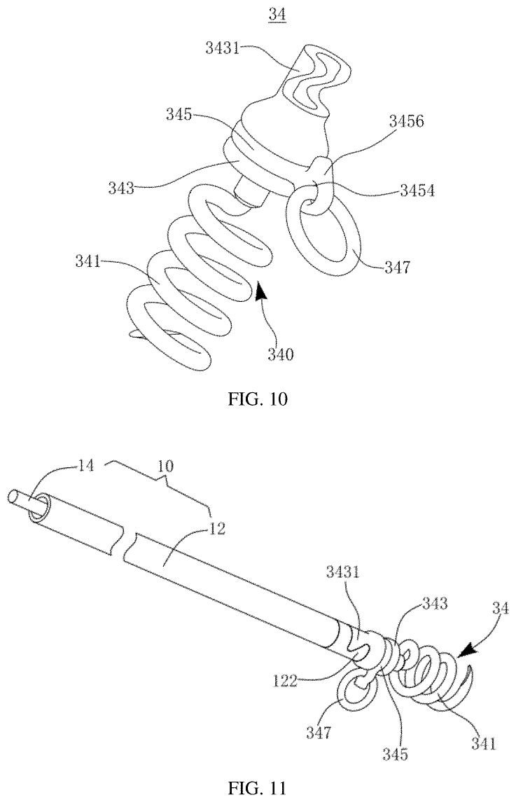

In the example of FIG. 9 and FIG. 10, the connecting member 345 defines a mounting aperture 3452 and a connecting aperture 3454. The anchoring base 343 is inserted into the mounting aperture 3452. Specifically, the connecting member 345 is movably sleeved on the anchoring base 343 through the mounting aperture 3452. The connecting aperture 3454 is configured to movably couple he threading ring 347, and the threading ring 347 passes through the connecting aperture 3454 of the connecting member 345, so as to be coupled with the connecting member 345, to realize a movable coupling between the threading ring 347 and the connecting member 345. The threading ring 347 can rotate about the central axis of the connecting aperture 3454 as the rotation axis. In the example of FIG. 9, the connecting aperture 3454 extends along the axial direction of the anchoring member 340, that is, the central axis of the connecting aperture 3454 is parallel to the central axis of the anchoring member 340. In the example of FIG. 10, the connecting aperture 3454 extends along the radial direction of the anchoring member 340, that is, the central axis of the connecting aperture 3454 is perpendicular to the central axis of the anchoring member 340.

In order to ensure the safety after implantation, the anchoring component 34 is made of biocompatible materials, including but not limited to metal materials (e.g., stainless steel) or polymer materials (e.g., PEEK, PET). The anchoring base 343 and the anchoring part 341 are preferably made of stainless steel with higher hardness. Both the connecting member 345 and the threading ring 347 can be made of stainless steel or polymer material.

Referring to FIGS. 11 to 13, the anchoring component 34 is detachably coupled to the distal end of the anchoring apparatus 10. Specifically, the anchoring apparatus 10 includes a driving tube 12 and a coupling rod 14 which is inserted into the driving tube 12. The proximal end of the anchoring base 343 includes a first coupling part 3431. A distal end of the driving tube 12 includes a second coupling part 122 detachably coupled with the first coupling part 3431. The coupling rod 14 is axially penetrated through the first coupling part 3431 and the second coupling part 122 that are mated with each other, such that the anchoring component 34 is coupled with the driving tube 12 of the anchoring apparatus 10, and the driving tube 12 is configured to drive the anchoring member 340 (i.e., the anchoring part 341) to anchor into the mitral annulus.

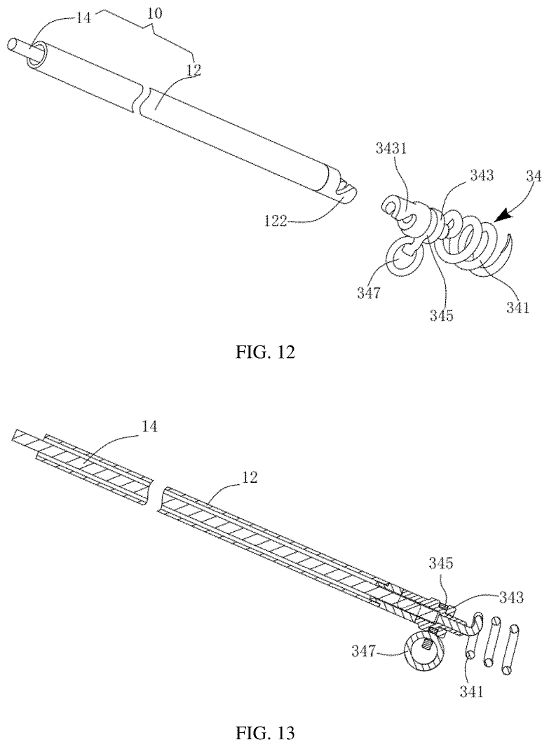

The first coupling part 3431 is a S-shaped buckle disposed at a proximal end of the anchoring base 343; the second coupling part 122 is a S-shaped buckles disposed at a distal end of the driving tube 12. Both the first coupling part 3431 and the second coupling part 122 each has an inner cavity. As shown in FIGS. 12 and 13, when the first coupling part 3431 and the second coupling part 122 are butted together, the respective S-shaped buckles of the anchoring base 343 and the driving tube 12 are engaged, and the inner cavities of the two are interconnected. The distal end of the coupling rod 14 inserted in the driving tube 12 protrudes from the distal end of the driving tube 12 and is inserted into the inner cavities of the first coupling part 3431 and the second coupling part 122, thereby restricting the separation of the first coupling part 3431 and the second coupling part 122, and keeping the anchoring component 34 being coupled to the driving tube 12. The anchoring member 340 can be rotated by rotating the driving tube 12, so that the anchoring part 341 is anchored into the mitral annulus. It can be understood that when the distal end of the coupling rod 14 is withdrawn from a butt joint of the first coupling part 3431 and the second coupling part 122, the first coupling part 3431 and the second coupling part 122 can be separated, thereby achieving the separation of the anchoring component 34 from the driving tube 12. The anchoring apparatus 10 can be made of metal materials or polymer materials, preferably metal material with higher hardness such as stainless steel.

In other embodiments, the first coupling part 3431 and the second coupling part 122 may be other structures, such as a matching structure between an engaging block and an engaging groove, which is not limited. The anchoring apparatus 10 may also include the driving tube 12 and a connecting tube sleeved on outside the driving tube 12. The distal end of the connecting tube is sleeved on outside the first coupling part 3431 and the second coupling part 122 which are mating connection, and can also play a role of preventing the first coupling part 3431 from separating the second coupling part 122.

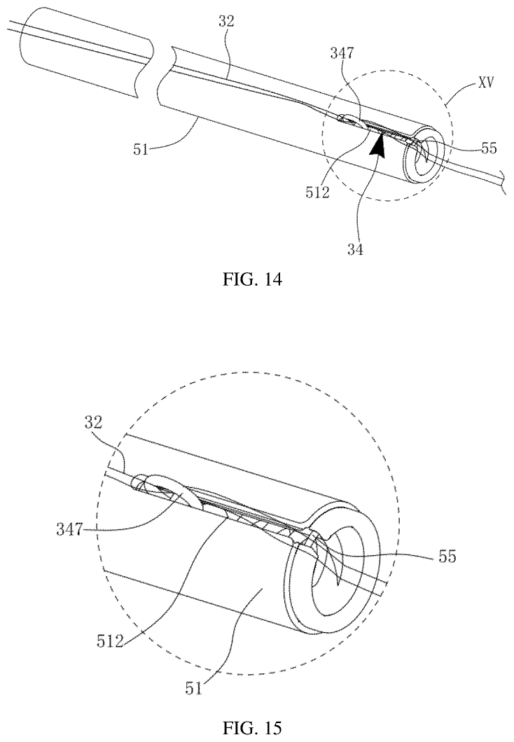

Referring to FIGS. 14 to 17, in some embodiments, a tube wall of the delivery sheath 51 defines a through slot 512 extending from a distal end of the delivery sheath 51 towards a proximal end of the delivery sheath 51. The through slot 512 interconnects with the lumen of the delivery sheath 51. The distal end of the through slot 512 defines an opening. Specifically, the through slot 512 extends to the distal end of the delivery sheath 51 in a line, so that the distal end of the through slot 512 has an opening, and the proximal end of the through slot 512 is closed. That is, the axial length of the through slot 512 is less than the axial length of the delivery sheath 51. In one example, the axial length of the through slot 512 is slightly greater than the axial length of the anchoring component 34. Of course, the through slot 512 can also extend to the proximal end of the delivery sheath 51, and the proximal end of the through slot 512 also has an opening.

Referring to FIGS. 15 and 17, the anchoring component 34 is movably inserted into the delivery sheath 51, and a part of the anchoring component 34 is exposed from the through slot 512. Each anchoring component 34 includes an anchoring member 340 and a threading structure disposed on the anchoring member 340. Specifically, the anchoring member 340 is received in the lumen of the delivery sheath 51, the threading structure is at least partially exposed from the through slot 512 and is located outside the delivery sheath 51, and the tightening string 32 is coupled to the threading structure the part of the threading structure outside the delivery sheath 51. The part of the tightening string 32 extending to the distal end from where it is coupled to the threading structure is located outside the delivery sheath 51. The tightening string 32 is separated from the anchoring member 340, which can prevent the tightening string 32 and the part of the anchoring component 34 located in the lumen of the delivery sheath 51 (i.e., the anchoring member 340) from winding, which is conducive to the implantation of the anchoring component 34.

Preferably, the tightening string 32 is coupled to the part of the threading structure located outside the delivery sheath 51, and the tightening string 32 is entirely located outside the delivery sheath 51. In this way, the radial dimension of the delivery sheath 51 can be reduced. Of course, the part of the tightening string 32 extending to the proximal end from where it is coupled to the threading structure may also be located in the lumen of the delivery sheath 51.

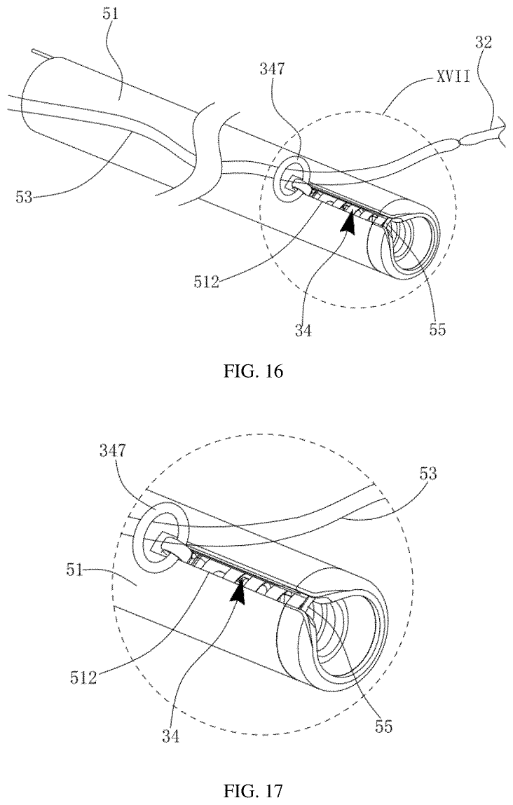

Preferably, the threading structure includes a connecting member 345 and a threading ring 347. When the anchoring component 34 is inserted into the delivery sheath 51, the anchoring member 340 is received in the lumen of the delivery sheath 51, and the threading structure enters into the through slot 512 of the delivery sheath 51 such that the threading ring 347 is at least partially exposed to the through slot 512, that is, the threading structure is partially located outside the delivery sheath 51. In the example of FIGS. 14 and 15, the connecting aperture 3454 of the connecting member 345 extends along the axial direction of the anchoring member 340; the threading ring 347 is partially exposed from the through slot 512 and is located outside the delivery sheath 51; the tightening string 32 is coupled to the part of the threading ring 347 outside the delivery sheath 51 and is integrally located outside the delivery sheath 51. In the example of FIGS. 16 and 17, the connecting aperture 3454 of the connecting member 345 extends along the radial direction of the anchoring member 340, the threading ring 347 is entirely located outside the delivery sheath 51, and the tightening string 32 is coupled to the threading ring 347 and is integrally located outside the delivery sheath 51.

Referring to FIGS. 9 and 10 again, in some embodiments, a holding end 3456 is protruded from one side of the connecting member 345 away from the anchoring member 340. A radial width of the holding end 3456 is adapted with a radial width of through slot 512 of the delivery sheath 51. When the anchoring component 34 is inserted into the distal end of the delivery sheath 51, the holding end 3456 is located in the through slot 512. In this way, a situation that rotating the connecting member 345 can drive the threading ring 347 to rotate can be prevented during the delivery of anchoring component 34, thereby ensuring stable and smooth delivery of the anchoring component 34. It should be noted that when the anchoring component 34 moves relative to the delivery sheath 51, the holding end 3456 moves in the through slot 512 along with the movement of the anchoring component 34 as a whole.

Referring again to FIGS. 14 to 17, in some embodiments, the delivery apparatus 50 further includes a blocking member 55. The blocking member 55 is movably arranged on the delivery sheath 51 and configured to close the distal opening of the through slot 512 to separate the tightening string 32 from anchoring component 34.

In some embodiments, the blocking member 55 extends along an axial direction of the delivery sheath 51. A distal end of the blocking member 55 is adapted to a circumference of the delivery sheath 51 to close a distal opening of the through slot 512. A proximal end of the blocking member 55 extends to the vicinity of the proximal end of the delivery sheath 51, making it convenient for the operator to operate the blocking member 55. For example, the proximal end of the blocking member 55 is exposed from the proximal end of the delivery sheath 51. The distal end of the blocking member 55 closes the distal opening of the through slot 512 when no external force is applied on the blocking member 55. In this way, due to a stop of the blocking member 55, the threading structure of the anchoring component 34 cannot slide out from the opening at the distal end of the through slot 512, thereby preventing the anchoring component 34 from being fall off the delivery sheath 51 during a delivery process, and ensuring that the anchoring member 340 is always in the delivery sheath 51 during the delivery process, to avoid entanglement of the tightening string 32 with the anchoring member 340. External force generally refers to the force applied on the blocking member 55 by the operator, such as a pulling force to move the blocking member 55 proximally, a pushing force to move the blocking member 55 distally, a rotational force to rotate the blocking member 55 about the central axis, or a force to deflect the blocking member 55 in the direction away from the central axis. That is, the blocking member 55 can be moved by pulling, pushing, rotating or deflecting.

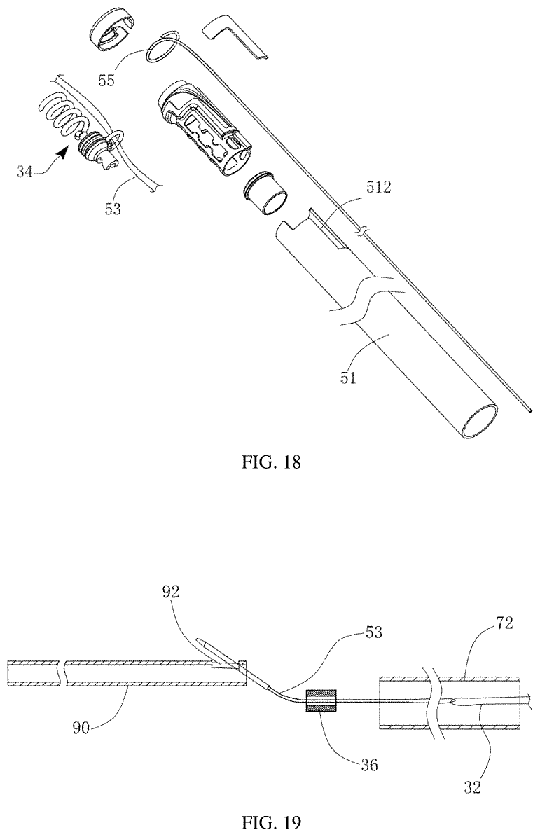

Specifically, please referring to FIG. 18, in one embodiment, at least the distal end of the blocking member 55 is made of shape memory materials such as but not limited to shape memory alloys, including nickel-titanium, nickel-titanium-based shape memory alloys, copper-based shape memory alloys, iron-based shape memory alloys, etc; and shape memory polymers, including high molecular weight polynorbornene, polycaprolactone, etc. That is to say, the distal end of the blocking member 55 is made of shape memory materials, or the blocking member 55 as a whole is made of shape memory materials. since the distal end of the blocking member 55 is made of shape memory materials, the distal end of the blocking member 55 has hyperelasticity. In another embodiment, at least the distal end of the blocking member 55 is made of elastic materials, such as stainless steel, beryllium bronze, etc. Therefore, the distal end of the blocking member 55 has the ability to restore from deformation. When the blocking member 55 is deformed by an external force, the distal end of the blocking member 55 restores its original state after the external force is removed.

Preferably, the distal end of the blocking member 55 is a non-closed loop in a natural state, that is the original state, so that the blocking member 55 has a good guidance and is easy to retract, which can increase the moving stroke of the blocking member 55 and ensure that the blocking effect of distal end of the blocking member 55 is formed by crossing the through slot 512, which closes the opening of the through slot 512 firmly and stably, and can ensure that the blocking member 55 has sufficient strength and is not easy to break. Of course, the distal end of the blocking member 55 may also include an arc-shaped segment adapted to the circumferential direction of the delivery sheath 51 in a natural state. It should be noted that since the distal end of the blocking member 55 is made of elastic materials or shape memory materials, the distal end of the blocking member 55 has elastic or hyperelastic property, so the natural state refers to that in this state, the distal end of the blocking member 55 maintains its shape without being affected by any external force. The radial cross-sectional shape of the blocking member 55 may be circular, oblate, rectangular, square, or other shapes, which are not specifically limited in the present disclosure. In one example, the blocking member 55 is an elongated blocking wire.

When the blocking member 55 is pulled proximally, the distal end of the blocking member 55 is moved away from the through slot 512, that is, the distal opening of the through slot 512 is opened. At this time, the anchoring component 34 can be inserted into the delivery sheath 51 or released from the delivery sheath 51. When the pulling force on the blocking member 55 is released, the distal end of the blocking member 55 restores its original state to close the distal opening of the through slot 512 again.

It can be understood that the distance that the blocking member 55 can be pulled and moved needs to be greater than the radial width of the through slot 512, so that the distal opening of the through slot 512 can be fully opened, so as not to prevent the threading structure from sliding out of the through slot 512. In addition, in order to prevent the blocking member 55 from being broken when pulled, the diameter of the blocking member 55 cannot be too small, and the diameter of the blocking member 55 is preferably 0.2 mm to 0.8 mm.

Referring to FIGS. 2 and 3, in some embodiments, the implant 30 further includes at least one spacer 36. Each spacer 36 is delivered along the delivery member 53 and is threaded onto the tightening string 32. The spacer 36 is located between the two anchoring components 34. A spacer 36 is located between at least two adjacent anchoring components 34, which can prevent excessive tightening of the tightening string 32, resulting in the short distance between the adjacent two anchoring components 34 and damage to the mitral annulus. At the same time, the spacer 36 can play a cushioning effect disperses the tightening force on the anchoring components 34 and ensures the stable implantation of the anchoring components 34. The spacer 36 is a cylindrical member with a certain length, preferably made of a biocompatible material. The spacer 36 may be wrapped with a membrane to facilitate endothelial climbing and reduce a risk of heart tissue, such as the mitral annulus, being damaged by the spacer 36.

Optionally, a spacer 36 may be disposed between any two adjacent anchoring components 34 of the plurality of anchoring components 34 of the implant 30 (as shown in FIG. 2 and FIG. 3), that is, the anchoring components 34 and the spacers 36 are alternately distributed. A spacer 36 may be provided every two or more anchoring components 34, that is, a spacer 36 is provided between partially two adjacent anchoring components 34, and there is no spacer 36 provided between other two adjacent anchoring components 36. This application does not limit this.

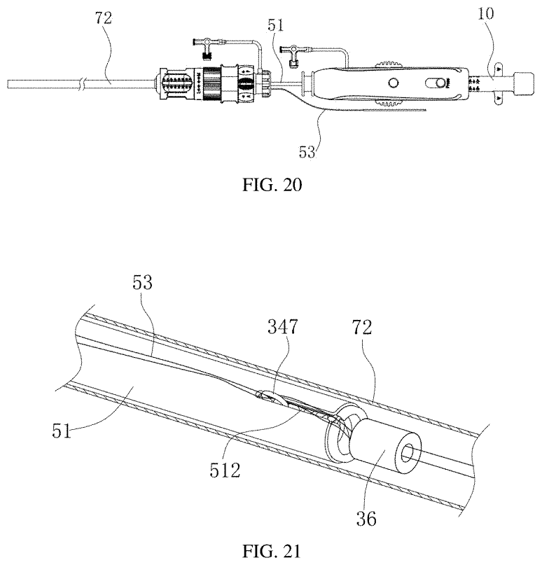

Referring to FIGS. 19-21, in some embodiments, the transcatheter annuloplasty system 1 further includes a push member 90 for pushing the spacer 36. Specifically, a distal end of the push member 90 defines a guide aperture 92. The guide aperture 92 can allow the delivery member 53 to pass through movably. After the proximal end of the delivery member 53 passes through the spacer 36 and the guide aperture 92 of the push member 90 in sequence, the push member 90 pushes the spacer 36, along the delivery member 53, into the delivery channel established by the guide apparatus 70, that is, a lumen of the second guide sheath 72. The delivery sheath 51 is inserted into the second guide sheath 72 to push the spacer 36 in the second guide sheath 72, so that the spacer 36 is delivered, along the delivery member 53, to the tightening string 32.

It can be understood that after the first anchoring component 34 is implanted into the mitral annulus, the delivery sheath 51 and the anchoring apparatus 10 are withdrawn. The spacer 36 is threaded onto a proximal end of the delivery member 53; after the delivery member 53 is passes through the guide aperture 92 of the push member 90, the push member 90 pushes the spacer 36, along the delivery member 53, into the second guide sheath 72 of the guide apparatus 70. Then, the push member 90 is withdrawn, the delivery sheath 51, equipped with the anchoring apparatus 10 and the second of the plurality of anchoring components 34 (hereinafter “second anchoring component 34”), is inserted into the second guide sheath 72. At this time, the delivery member 53 passes through the threading structure of the second anchoring component 34, the spacer 36 is located on one side of the distal end of the delivery sheath 51. As a result, the delivery sheath 51 is moved distally in the axial direction, within the second guide sheath 72, that is, the spacer 36 and the second anchoring component 34 are delivered, along the delivery member 53, so as to be threaded onto the tightening string 32, and push the spacer 36 to the mitral annulus. The anchoring apparatus 10 then drives the second anchoring component 34 out of the delivery sheath 51 and implants the second anchoring component 34 into the mitral annulus such that the spacer 36 is located between the two anchoring components 34. The same steps are repeated to sequentially implant a plurality of anchoring components 34 into the mitral annulus while the spacer 36 is located between each two or more anchoring components 34 in sequence. The distance between two adjacent anchoring components 34 needs to be greater than the axial length of the spacer 36.

Referring again to FIGS. 1 to 3, in some embodiments, the implant 30 further includes a retractor 38 for adjusting and locking the tightening string 32. The transcatheter annuloplasty system 1 further includes an adjustment apparatus 80 for delivery and controlling the retractor 38. Specifically, after the plurality of anchoring components 34 and spacers 36 are implanted into the mitral annulus, the retractor 38 is threaded, along the delivery member 53, onto the tightening string 32, and then the adjustment apparatus 80 is configured to drive the retractor 38 so that the tightening string 32 is wrapped around the retractor 38, so as to retract the tightening string 32. The tightening string 32 is tightened, thereby contracting the spacing of the plurality of anchoring components 34 to contract the size of the mitral annulus. When the retractor 38 stops winding the tightening string 32, the tightening string 32 is locked by the retractor 38 so that the tightening string 32 is held on the mitral annulus. It can be understood that, the string retractor 38 can be fitted on the tightening string 32 through the delivery member 53 and the tightening string 32 can be released smoothly without the need to deliver the retractor 38 into the patient in advance, which simplifies the operation process, reduces the difficulty of the operation, and shortens the operation time.

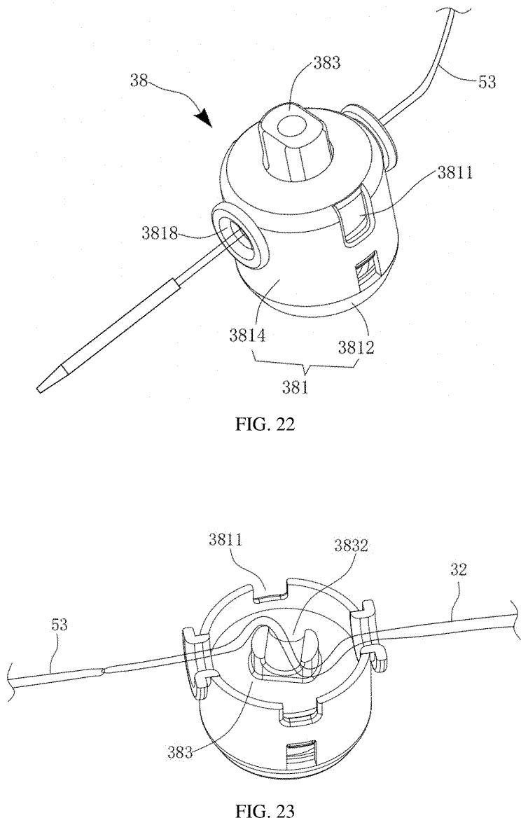

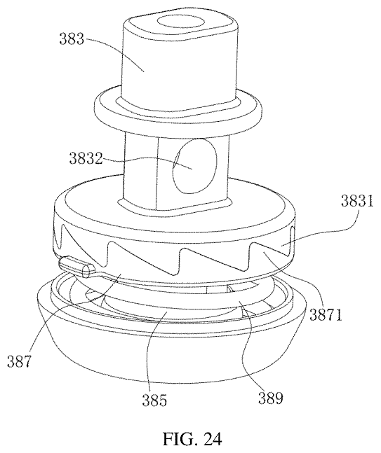

Referring to FIGS. 22 to 25, the retractor 38 includes a casing 381 and a spool 383 rotatably disposed in the casing 381. Since the delivery member 53 is coupled with the tightening string 32, the retractor 38 can be fitted on the delivery member 53 and move towards the distal end of the delivery member 53 so that the proximal end of the tightening string 32 can movably pass through the casing 381 and the spool 383. The spool 383 rotates relative to the casing 381 to wind the tightening string 32. When the spool 383 stops rotating, the tightening string 32 is locked in a radial space between the spool 383 and the casing 381.

It can be understood that the tightening string 32 can be wound by controlling the spool 383 to rotate relative to the casing 381, so that the tightening string 32 is continuously tightened to contract the spacing of the plurality of anchoring components 34 and the mitral annulus is contracted until the blood regurgitation is decreased or disappeared to stop rotating the spool 383. At this time the tightening string 32 is locked in the radial space between the spool 383 and the casing 381, and the tightening string 32 maintains a certain length on the mitral annulus. The tightening string 32 is wound and locked by the retractor 38, and the tightening effect of the tightening string 32 is good. Furthermore, if after a period of time, the recurrence of regurgitation due to the mitral annulus dilatation of the patient, the retractor 38 can be directly controlled to further wind the tightening string 32 to contract the mitral annulus to decrease or disappear the regurgitation, thereby avoiding the secondary operation which caused greater damage to the patient. The retractor 38 may be made of biocompatible materials, such as stainless steel, which is not limited.

It should be noted that the tightening string 32 is wrapped around the spool 383 for at least two turns, preferably at least three turns, and the friction between each turn of the tightening string 32 can offset the pulling force generated by the valve movement, ensuring that the tightening string 32 is not pulled by the valve movement, and the tightening string 32 maintains a certain length on the mitral annulus.

Specifically, the casing 381 includes a bottom case 3812 and an outer case 3814. Both the proximal end and the distal end of the outer case 3814 have openings. The bottom case 3812 is fixedly coupled to the distal end of the outer case 3814 to form a mounting space 3816. The retractor 38 further includes a limit post 385, an anti-rotation wheel 387 and an elastic member 389. The limit post 385, the elastic member 389, the anti-rotation wheel 387, and the spool 383 are arranged in the mounting space 3816 of the casing 381.

The spool 383 defines a through aperture 3832 along its radial direction. The casing 3814 defines two string apertures 3818 on both sides of the spool 383. Preferably, the central axes of the two string apertures 3818 and the central axis of the through aperture 3832 are in a same plane. The spool 383 can be rotated to make the central axis of the through aperture 3832 and the central axes of the two string apertures 3818 be collinear, which is convenient for the delivery member 53 passing smoothly through the two string apertures 3818 and the through aperture 3832, so as to deliver the retractor 38 onto the tightening string 32.

The distal end of the limit post 385 is fixedly coupled with the bottom case 3812. The anti-rotation wheel 387 is axially movably sleeved on the limit post 385 and can not rotate relative to the limit post 385. The elastic member 389 abuts between the bottom case 3812 and the anti-rotation wheel 387. The spool 383 is rotatably sleeved on the limit post 385. The limit post 385 and the proximal end of the casing 3814 cooperatively limit an axial displacement of the spool 383 in the mounting space, so that the spool 383 can only rotate. The proximal surface of the anti-rotation wheel 387 includes a plurality of first oblique teeth 3871 along the circumferential direction, and the distal surface of the spool 383 defines a plurality of second oblique teeth 3831, along the circumferential direction, which can only rotate unidirectionally relative to the first oblique teeth 3871. The elastic member 389 is configured to provide elastic force to the anti-rotation wheel 387, so that the first oblique teeth 3871 of the anti-rotation wheel 387 engages the second oblique teeth 3831 of the spool 383.

When the spool 383 rotates forward relative to the casing 381 and the anti-rotation wheel 387, the second oblique teeth 3831 slips on the first oblique teeth 3871 to move the anti-rotation wheel 387 distally. When the spool 383 rotates forward relative to the anti-rotation wheel 387 to an angle of one oblique tooth, after receiving the elastic force given by the elastic member 389, the anti-rotation wheel 387 will move proximally, so that the first oblique teeth 3871 and the second oblique teeth 3831 are re-engaged, and the spool 383 can continue to rotate relative to the casing 381 and the anti-rotation wheel 387. When the spool 383 is rotated in the reverse direction, due to the obstruction of the first oblique teeth 3871, the second oblique teeth 3831 cannot move the anti-rotation wheel 387 distally, so that the spool 383 cannot be reversed. Therefore, when the spool 383 stops rotating, the tightening string 32 is locked in the radial space between the spool 383 and the casing 381.

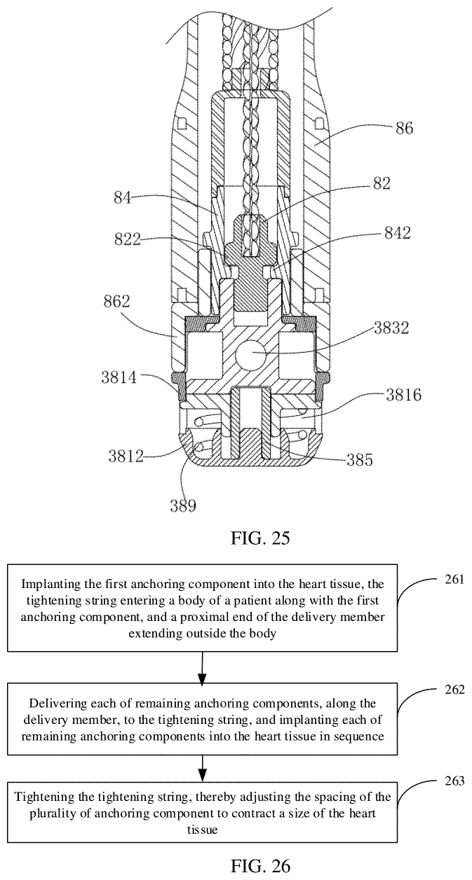

Please refer to FIG. 1 and FIG. 25 together. In some embodiments, the adjusting apparatus 80 includes a threading rod 82, a rotating tube 84 and an outer sheath 86, which are arranged from inside to outside. The outer sheath 86 is clamped with the casing 381 of the retractor 38 to restrict the rotation relative to the casing 381. The rotating tube 84 is configured to sleeve with the proximal end of the spool 383 and the rotating tube 84 and the spool 383 cannot rotate relative to each other. The threading rod 82 is configured to threaded couple with the spool 383 to maintain the coupling of the rotating tube 84 with the spool 383. Therefore, by rotating the rotating tube 84, the spool 383 can be driven to rotate to wind and tighten the tightening string 32.

In some embodiments, the outer case 3814 of the casing 381 defines a clamping groove 3811. A distal end of the outer sheath 86 includes a clamping claw 862 corresponding to the clamping groove 3811. The outer sheath 86 is coupled to the casing 381 through the cooperation between the claw 862 and the groove 3811. The proximal end of the spool 383 protrudes from an opening of the proximal end of the casing 3814. The proximal end of the spool 383 defines a threaded aperture along its axial direction. The inner wall of the rotating tube 84 is protruded with a first bump 842, and the outer wall of the threading rod 82 is protruded with a second bump 822. After the rotating tube 84 is sleeved with the spool 383, the threading rod 82 is screwed with the threaded aperture, that is, the threading rod 82 is screwed with the spool 383, so that the second bump 822 presses the first bump 842 on the spool 383 in order to limit the proximal movement of the rotating tube 84, the rotating tube 84 and the spool 383 can remain coupled. At this time, the outer sheath 86 restricts the rotation of the casing 381, and drives the rotating tube 84 and the threading rod 82 to rotate synchronously, thereby driving the spool 383 to rotate relative to the casing 381 to wind and tighten the tightening string 32, so as to contract the mitral annulus. Of course, the rotating tube 84 can also be driven to rotate independently to drive the spool 383 to rotate, and the threading rod 82 will follow the spool 383 to passively rotate.

In other embodiments, the delivery apparatus 50 may omit the delivery member 53, and the tightening string 32 may be long enough to extend outside the body of patient. After the plurality of anchoring components 34 are implanted, the tightening string 32 is tightened to contract the mitral annulus to achieve a better effect of reducing blood regurgitation, the tightening string 32 can be locked by a knot locking device or pin locking device. Alternatively, the tightening string 32 can be tightened by the retractor 38, and after achieving the better effect of reducing blood regurgitation, the redundant tightening string 32 can be cut.

Referring to FIG. 26, a method for contracting heart tissue using a transcatheter annuloplasty system 1 is illustrated, according to one embodiment of the present disclosure. The transcatheter annuloplasty system 1 includes a plurality of anchoring components 34, a tightening string 32 and a delivery member 53. Both the tightening string 32 and the delivery member 53 are flexible. A distal end of the tightening string 32 is coupled to a first anchoring component 34, and a distal end of the delivery member 53 is coupled to a proximal end of the tightening string 32. The method for contracting heart tissue using the transcatheter annuloplasty system 1 include block 261, block 262 and block 263.

Block 261: implanting the first anchoring component 34 into the heart tissue, the tightening string 32 entering a body of a patient along with the first anchoring component 34, and a proximal end of the delivery member 53 extending outside the body.

Block 262: delivering each of remaining anchoring components 34, along the delivery member 53, to the tightening string 32, and implanting each of remaining anchoring components 34 into the heart tissue in sequence.

Block 263: tightening the tightening string 32, thereby adjusting the spacing of the plurality of anchoring component 34 to contract a size of the heart tissue.

In the method for contracting heart tissue using the transcatheter annuloplasty system 1 provided in the present disclosure, the anchoring components 34 are delivered, along the delivery member 53, to the tightening string 32, and after the plurality of anchoring components 34 are implanted into the heart tissue, the tightening string 32 is tightened to adjust the spacing of the plurality of anchoring components 34, thereby achieving a purpose of contracting a size of the heart tissue. In this way, an appropriate implanting length of the tightening string 32 can be selected, so that it is not necessary to cut the tightening string 32 in the body, thereby avoiding the shedding of the string particles caused by the tightening string 32, and the operation is safer. It should be noted that the heart tissue is one of the mitral annulus, the tricuspid annulus, the left ventricle wall, and the right ventricle wall.

The plurality of anchoring components 34 which coupled in series by the tightening string 32 can be implanted into the mitral annulus or the tricuspid annulus to directly contract the annulus, and implanted into the left ventricular wall or the right ventricular wall for contracting the ventricle to reduce a volume of the ventricle, so as to achieve a purpose of annuloplasty, thereby achieving a treatment of mitral or tricuspid regurgitation. Furthermore, volume reduction of the left ventricle can achieve a treatment of heart failure caused by abnormal left ventricular function.

In some embodiments, the method further includes separating the delivery member 53 from the tightening string 32, and withdrawing the delivery member 53.

Specifically, it can be seen from the above description that the distal end of the delivery member 53 is coupled to the proximal end of the tightening string 32 in a U-shape. The delivery member 53 can be directly pulled from the first loop 321 of the tightening string 32, and then withdrawn the delivery member 53 to outside the body, which is easy to separate the delivery member 53 from the tightening string 32.

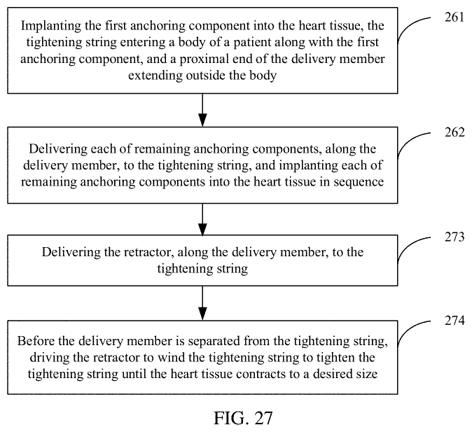

Optionally, in one embodiment, the transcatheter annuloplasty system 1 further includes a retractor 38 for winding the tightening string 32 to tighten the tightening string 32. When the retractor 38 stops winding the tightening string 32, the tightening string 32 is locked by the retractor 38. Specifically, as can be seen from the above description, the retractor 38 includes a casing 381 and a spool 383 rotatably disposed in the casing 381. There is also an anti-rotation mechanism within the casing 381, which allows the spool 383 to rotate, in a forward direction relative to the casing 381, to wind the tightening string 32 passing through the retractor 38, and restricts the spool 383 from rotating in an opposite direction relative to the casing 381. Therefore, when the rotation of the spool 383 is stopped, the tightening string 32 can be locked. Please referring to FIG. 27, the block 263 specifically includes block 273 and block 274.

Block 273: delivering the retractor 38, along the delivery member 53, to the tightening string 32.

Block 274: before the delivery member 53 is separated from the tightening string 32, driving the retractor 38 to wind the tightening string 32 to tighten the tightening string 32 until the heart tissue contracts to a desired size.

Therefore, separating the delivery member 53 from the tightening string 32 after the tightening string 32 is wrapped around the retractor 38 to contract the heart tissue to the desired size. It should be noted that a coupled part of the delivery member 53 and the tightening string 32 should not be wound into the casing 381 of the retractor 38. Contraction of heart tissue to the desired size, which means that the valve annulus is contracted to a size that eliminates or relieves a symptoms of regurgitation to a desired effect; or means that the ventricles is contracted to a size that eliminates or relieves a symptoms of regurgitation to a desired effect, or means that the size of ventricle is reached to treat heart failure.

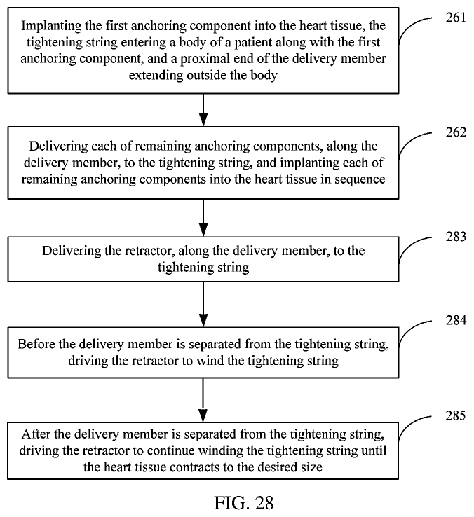

Optionally, in another embodiment, please referring to FIG. 28, the block 263 specifically includes block 283, block 284 and block 285.

Block 283: delivering the retractor 38, along the delivery member 53, to the tightening string 32.

Block 284: before the delivery member 53 is separated from the tightening string 32, driving the retractor 38 to wind the tightening string 32.

Block 285: after the delivery member 53 is separated from the tightening string 32, driving the retractor 38 to continue winding the tightening string 32 until the heart tissue contracts to the desired size.

It can be understood that the retractor 38 is delivered to the predetermined position of the tightening string 32 through the delivery member 53. Since the valve movement will produce tension on the tightening string 32, the coupling between the retractor 38 and the tightening string 32 is not stable. Therefore, in order to avoid the tightening string 32 from being pulled to cause the retractor 38 to separate from the tightening string 32, before the delivery member 53 is separated from the tightening string 32, the retractor 38 needs to wind the tightening string 32. Specifically, the retractor 38 winds the tightening string 32 at least two turns (preferably at least three turns), so that the frictional force between each turn of the tightening string 32 can offset the pulling force generated by the valve movement, ensuring that the tightening string 32 will not be pulled and separated from the retractor 38, and then withdraw the delivery member 53, and continue to wind the tightening string 32 until the heart tissue contracts to the desired size. In this way, an appropriate implanting length of the tightening string 32 is selected, and the proximal end of the tightening string 32 can be completely retracted into the casing 381 of the retractor 38, which can prevent a extra part of the tightening string 32 from floating in the atrium or ventricle after surgery, which increases risk of postoperative period.

Furthermore, the transcatheter annuloplasty system 1 further includes an adjustment apparatus 80 for delivery and controlling the retractor 38. Driving the retractor 38 to wind the tightening string 32, includes: driving the adjustment apparatus 80 to drive the retractor 38 so as to wind the tightening string 32.

Specifically, the outer sheath 86 of the adjustment apparatus 80 is clamped with the casing 381 of the retractor 38, the rotating tube 84 is sleeved with the proximal end of the spool 383, and the threading rod 82 is threadedly coupled with the proximal end of the spool 383. The outer sheath 86 remains stationary, and the rotating tube 84 and the threading rod 82 are driven to rotate synchronously, which can drive the spool 383 to rotate relative to the casing 381 to wind and tighten the tightening string 32.

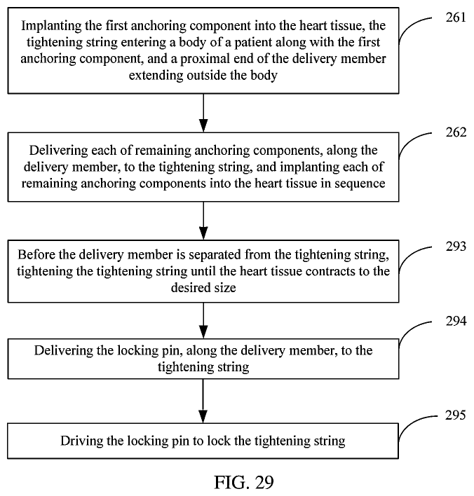

Optionally, in another embodiment, the transcatheter annuloplasty system 1 further includes a locking pin. Referring to FIG. 29, the block 263 specifically includes block 293, block 294 and block 295.

Block 293: before the delivery member 53 is separated from the tightening string 32, tightening the tightening string 32 until the heart tissue contracts to the desired size.

Block 294: delivering the locking pin, along the delivery member 53, to the tightening string 32.

Block 295: driving the locking pin to lock the tightening string 32.

Specifically, after the tightening string 32 is tightened, the locking pin is delivered to the tightening string 32, and the tightening string 32 can be locked by pressing and deforming the locking pin. Since the locking pin cannot pass through the threading structure of the anchoring components 34 or a lumen of the spacer 36, the tightening string 32 was able to maintain the desired size of the heart tissue.

In some embodiments, the transcatheter annuloplasty system 1 further includes at least one spacer 36, and the method further includes: delivering the at least one spacer 36 in sequence, along the delivery member 53, to the tightening string 32, and one of the at least one spacer 36 being provided between at least two adjacent anchoring components 34.

It can be understood that disposing the spacer 36 between the two anchoring components 34 can prevent the tightening string 32 from being excessively tightened and the distance between the two adjacent anchoring components 34 from being too short to damage the heart tissue. At the same time, the spacer 36 can play a buffering role, to disperse the tightening force received by the anchoring component 34 to ensure the stable implantation of the anchoring component 34.

In some embodiments, the transcatheter annuloplasty system 1 further includes a delivery sheath 51, the anchoring component 34 is movably inserted therein. Delivering the at least one spacer 36 in sequence, along the delivery member53, to the tightening string 32, includes follow steps.

After the proximal end of the delivery member 53 being passed through one of the at least one spacer 36, pushing the spacer 36 to be delivered along the delivery member 53.

Abutting a distal end of the delivery sheath 51 to the spacer 36.

Pushing the delivery sheath 51 to move distally to push the spacer 36 to continue moving, along the delivery member 53, until the spacer 36 is delivered onto the tightening string 32.

It will be appreciated that the delivery sheath 51 is configured to deliver the anchoring component 34 to the heart tissue. While using the delivery sheath 51 to deliver the anchoring component 34, the spacer 36 is pushed, along the delivery member 53, to move to the tightening string 32, that is, the anchoring component 34 and the spacer 36 are simultaneously delivered, which can reduce the operation time and improve the efficiency.

In some embodiments, the transcatheter annuloplasty system 1 further includes a delivery sheath 51 and an anchoring apparatus 10. The anchoring component 34 is detachably coupled to a distal end of the anchoring apparatus 10 and inserted into the delivery sheath 51. The anchoring apparatus 10 is configured to drive the anchoring components 34 to be implanted into heart tissue.

Specifically, block 251 includes following steps.

Detachably coupling the first anchoring component 34 to the distal end of the anchoring apparatus 10 and inserting the first anchoring component 34 into the delivery sheath 51.

Pushing the delivery sheath 51 until the distal end of the delivery sheath 51 abuts the heart tissue.

Driving the anchoring apparatus 10 to drive the first anchoring component 34 to be implanted into heart tissue.

Specifically, block 252 includes following steps.

Detachably coupling one of the remaining anchoring components 34 to the distal end of the anchoring apparatus 10 and inserting the one of the remaining anchoring components 34 into the delivery sheath 51.

Coupling the anchoring component 34 to the proximal end of the delivery member 53.

Pushing the delivery sheath 51 until the distal end of the delivery sheath 51 abuts the heart tissue, the anchoring component 34 is delivered, along with a pushing of the delivery sheath 51, along the delivery member 53, to the tightening string 32.

Driving the anchoring apparatus 10 to drive the anchoring component 34 to be implanted into the heart tissue.

In some embodiments, the anchoring component 34 include an anchoring member 340 for anchoring into the heart tissue and a threading structure for coupling the tightening string 32. Specifically, the anchoring member 340 is rotated to anchor into the heart tissue, and the threading structure can be rotatably sleeved on the anchoring member 340. The threading structure includes a connecting member 345 and a threading ring 347. The connecting member 345 is rotatably sleeved on the anchoring member 340, the threading ring 347 is movably coupled to the connecting member 345. The threading ring 347 is configured to couple the tightening string 32.

In some embodiments, the tube wall of the delivery sheath 51 defines a through slot 512 extending from a distal end of the delivery sheath 51 towards a proximal end of the delivery sheath 51. The through slot 512 interconnects with the lumen of the delivery sheath 51. The distal end of the through slot 512 defines an opening. The method further includes: after the anchoring component 34 is detachably coupled to the distal end of the anchoring apparatus 10, withdrawing the anchoring apparatus 10 proximally so that the anchoring member 340 is received into the lumen of the delivery sheath 51, and the threading structure is at least partially located outside the delivery sheath 51, so that the tightening string 32 is separated from the anchoring member 340.

Specifically, as described above, the anchoring apparatus 10 is inserted into the lumen of the delivery sheath 51. When coupling the anchoring component 34 to the anchoring apparatus 10, the distal end of the anchoring apparatus 10 needs to be pushed out of the delivery sheath 51 to expose the second coupling part 122 of the driving tube 12. The first coupling part 3431 of the anchoring components 34 is coupled to the second coupling part 122. The coupling between the first coupling part 3431 and the second coupling part 122 is maintained by the coupling rod 14 or the connecting pipe, so as to maintain the coupling between the anchoring component 34 and the driving tube 12. After the anchoring component 34 is coupled to the anchoring apparatus 10, the anchoring apparatus 10 is withdrawn proximally to drive the anchoring component 34 to be inserted into the delivery sheath 51, so that the anchoring member 340 is received into the lumen of the delivery sheath 51, and the threading structure enters the through slot 512, such that the threading structure is at least partially outside the delivery sheath 51. Therefore, the tightening string 32 is coupled to the part of the threading structure outside the delivery sheath 51, so that the tightening string 32 is separated from the anchoring member 340, which can prevent the tightening string 32 from being entangled with the anchoring member 340, which is beneficial to the successful implantation of the anchoring components 34.

In some embodiments, the transcatheter annuloplasty system 1 further includes a blocking member 55. The proximal end of the blocking member 55 extends along an axial direction of the delivery sheath 51. The distal end of the blocking member 55 is adapted to a circumferential direction of the delivery sheath 51 to close the opening of the through slot 512. The method also includes: before the anchoring apparatus 10 is withdrawn proximally, pulling the blocking member 55 proximally to open the opening of the through slot 512; and after the anchoring apparatus 10 is withdrawn proximally, releasing the blocking member 55 so that the blocking member 55 closes the opening of the through slot 512.

It can be understood that the blocking member 55 is pulled proximally to open the opening of the through slot 512, so that the threading structure of the anchoring components 34 can enter the through slot 512; then the blocking member 55 is released so that the blocking member 55 closes the opening of the through slot 512, the threading structure of the anchoring component 34 cannot slide out from the distal opening of the through slot 512, so that the anchoring component 34 can be prevented from fall off the delivery sheath 51 during the delivery process.

In some embodiments, the method further includes: after the anchoring components 34 is implanted into the heart tissue, pulling the blocking member 55 proximally to open the opening of the through slot 512 to release the anchoring component 34 from the delivery sheath 51.

It can be understood that, after the anchoring component 34 is implanted into the heart tissue, that is, after the anchoring part 341 of the anchoring member 340 is anchored into the heart tissue, opening the opening of the through slot 512, so that the threading structure of the anchoring component 34 can slide out the through slot 512. The delivery sheath 51 is withdrawn proximally to release the anchoring component 34 from the delivery sheath 51. In other embodiments, when the distal end of the delivery sheath 51 reaches a predetermined treatment site, the blocking member 55 can be pulled proximally to open the opening of the through slot 512, and then the anchoring apparatus 10, inserted into the delivery sheath 51, can driver the anchoring component 34 out of the distal end of the delivery sheath 51 and to be implanted into the heart tissue, the threading structure moves in the through slot 512, with the movement of the anchoring member 340; the delivery sheath 51 can be withdrawn proximally so that the anchoring component 34 can be released from delivery sheath 51.

In some embodiments, the transcatheter annuloplasty system 1 further includes a guide apparatus 70 having a channel. The method further includes: before the first anchoring component 34 is implanted into the heart tissue, establishing a delivery pathway from outside the body to heart tissue through the guide apparatus 70.

Therefore, the delivery sheath 51, the adjustment apparatus 80, and the like can enter inside the body of the patient through the delivery pathway.

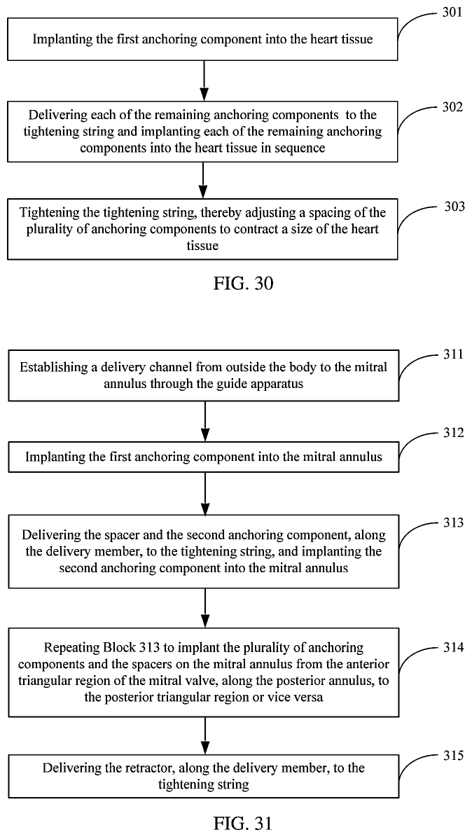

Referring to FIG. 30, another method for contracting heart tissue using the transcatheter annuloplasty system 1 is provided, according to one embodiment of the present disclosure. The transcatheter annuloplasty system 1 includes a plurality of anchoring components 34, a tightening string 32 and a delivery sheath 51. The tightening string 32 is flexible. A distal end of the tightening string 32 is coupled to the first anchoring component 34. The tube wall of the delivery sheath 51 defines a through slot 512 extending from the distal end towards the proximal end. A part of the anchoring component 34 is exposed from the through slot 512. The method for contracting heart tissue using the transcatheter annuloplasty system 1 includes block 301, block 302 and block 303.

Block 301: implanting the first anchoring component 34 into the heart tissue.

Block 302: delivering each of the remaining anchoring components 34 to the tightening string 32 and implanting each of the remaining anchoring components 34 into the heart tissue in sequence.

Block 303: tightening the tightening string 32, thereby adjusting a spacing of the plurality of anchoring components 34 to contract a size of the heart tissue.

In the method for contracting heart tissue using the transcatheter annuloplasty system 1 provided in the present disclosure, the anchoring components 34 are delivered to the heart tissue through the delivery sheath 51, and after the plurality of anchoring components 34 are implanted into the heart tissue, the tightening string 32 are tightened to contract the spacing of the plurality of anchoring components 34, for the purpose of contracting the size of the heart tissue. The part of the anchoring component 34 is exposed from the through slot 512 of the delivery sheath 51 so that the tightening string 32 can be coupled to the part of the anchoring component 34 located outside the delivery sheath 51. The part of the tightening string 32 exending to the distal end from where it couples the anchoring component 34 is located outside the delivery sheath 51, which can prevent the tightening string 32 from being entangled with the part of the anchoring component 34 inserted into the lumen of the delivery sheath 51, which is beneficial to the implantation of the anchoring component 34. It should be noted that the heart tissue is one of the mitral annulus, the tricuspid annulus, the left ventricle wall, and the right ventricle wall.

The plurality of anchoring components 34, coupled in series by the tightening string 32, can be implanted into the mitral annulus or the tricuspid annulus to directly contract the valve annulus, or implanted into the left ventricular wall or the right ventricular wall, to contract the ventricular wall and reduce the volume of the ventricle, thereby achieving the treatment of mitral regurgitation or tricuspid regurgitation. Furthermore, the volume reduction of the left ventricle can also treat heart failure caused by abnormal left ventricular function. The method of tightening the tightening string 32 can be the method of winding the tightening string 32 by using the above-mentioned retractor 38 or the method of tightening the tightening string 32 and locking it with a locking pin. After the tightening string 32 is tightened and locked, the excess part can be cut off.

In some embodiments, the transcatheter annuloplasty system 1 further includes an anchoring apparatus 10 for driving the anchoring component 34 to be implanted into the heart tissue. The anchoring component 34 includes an anchoring member 340 and a threading structure. The anchoring member 340 is configured to be anchored into the heart tissue; the threading structure is configured to couple the tightening string 32. The distal end of the through slot 512 defines an opening. The method further includes: detachably coupling the anchoring component 34 to the distal end of the anchoring apparatus 10 and inserting the anchoring component 34 into the delivery sheath 51, so that the anchoring member 340 is received in the lumen of the delivery sheath 51, and the threading structure enters the through slot 512 and a part of the threading structure is located outside the delivery sheath 51.

Specifically, as described above, the anchoring apparatus 10 is inserted into the lumen of the delivery sheath 51; the distal end of the anchoring apparatus 10 protrudes from the distal end of the delivery sheath 51 and is coupled to the anchoring component 34, and then withdrawing the anchoring apparatus 10 proximally drives the anchoring component 34 to be inserted into the delivery sheath 51, so that the anchoring member 340 is received in the lumen of the delivery sheath 51, and the threading structure enters the through slot 512 and is partially located outside the delivery sheath 51. Therefore, the tightening string 32 is coupled to the part of the threading structure outside the delivery sheath 51, so that the tightening string 32 is separated from the anchoring member 340, which can prevent the tightening string 32 from being entangled with the anchoring member 340, which is conducive to the implantation of the anchoring component 34.

In some embodiments, the transcatheter annuloplasty system 1 further includes a blocking member 55. The blocking member 55 extends along the axial direction of the delivery sheath 51. A proximal end of the blocking member 55 extends to the vicinity of the proximal end of the delivery sheath 51, and a distal end of the blocking member 55 is adapted to a circumferential direction of the delivery sheath 51 to close the opening of the through slot 512. The method includes: before the anchoring component 34 is inserted into the delivery sheath 51, pulling the blocking member 55 proximally to open the opening of the through slot 512; after the anchoring component 34 is inserted into the delivery sheath 51, releasing the blocking member 55 so that the blocking member 55 closes the opening of the through slot 512.

It can be understood that the blocking member 55 is pulled proximally to open the opening of the through slot 512, so that the threading structure of the anchoring component 34 can enter the through slot 512; the blocking member 55 is then released so that the blocking member 55 closes the opening of the through slot 512. As the blocking member 55 prevents the threading structure of the anchoring component 34 from sliding out the distal opening of the through slot 512, thereby preventing the anchoring component 34 fall off from the delivery sheath 51 during a delivery process.

In some embodiments, the method further includes: after the anchoring component 34 being implanted into the heart tissue, pulling the blocking member 55 proximally to open the opening of the through slot 512 to release the anchoring component 34 from the delivery sheath 51.

It can be understood that, after the anchoring component 34 is implanted into the heart tissue, that is, after the anchoring part 341 of the anchoring member 340 is anchored into the heart tissue, opening the opening of the through slot 512, so that the threading structure of the anchoring component 34 can slide out the through slot 512. The delivery sheath 51 is withdrawn proximally to release the anchoring component 34 from the delivery sheath 51.

In some embodiments, the transcatheter annuloplasty system 1 further includes at least one spacer 36. The method further includes: delivering the at least one spacer 36 to the tightening string 32 in sequence; at least two adjacent anchoring components 34 is provided with a spacer 36 therebetween.

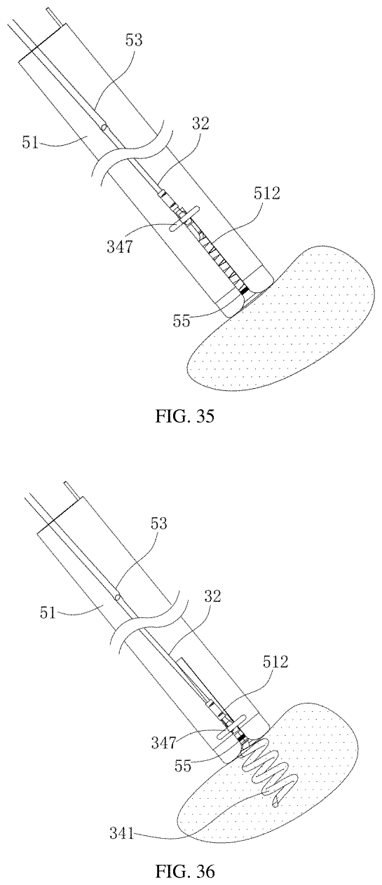

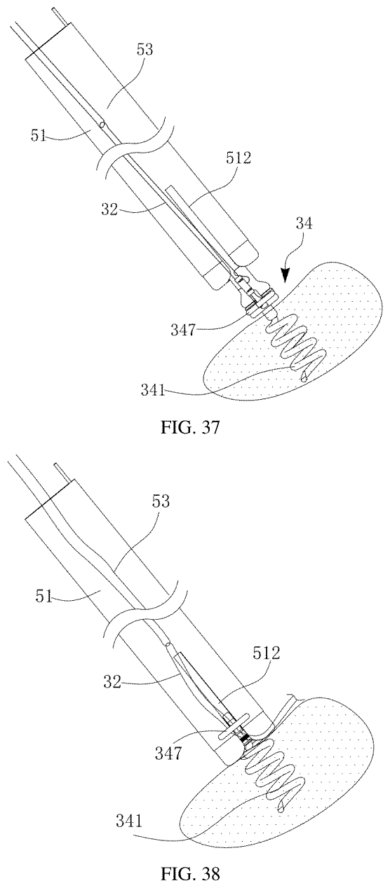

Referring to FIG. 21 and FIGS. 31 to 40, the method for contracting the annulus using the transcatheter annuloplasty system 1, according to one embodiment of the present disclosure, will be described by taking the transcatheter annuloplasty system 1 of the present disclosure to the mitral valve annuloplasty as an example. The intervention path is: femoral vein-inferior vena cava-right atrium (RA)-atrial septum (AS)-left atrium (LA)-mitral annulus (MA).

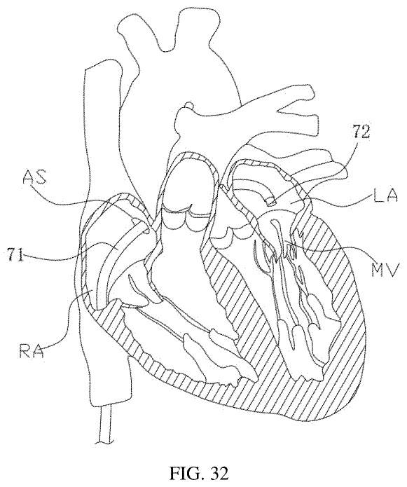

Block 311: please referring to FIG. 32, establishing a delivery channel from outside the body to the mitral annulus through the guide apparatus 70.

Specifically, through a femoral vein puncture, a track of the femoral vein-inferior vena cava-right atrium-atrial septum-left atrium-mitral annulus is established by a transseptal puncturing device and a guide wire. Advancing the first guide sheath 71 and the second guide sheath 72, along the guide wire, until its distal end passes through the fossa ovalis to the left atrium and near the mitral annulus, and then withdrawning the transseptal puncturing device and the guide wire. Under the guidance of the medical image device, the distal end of the guide apparatus 70 is adjusted to the vicinity of the mitral annulus and maintained in this state.

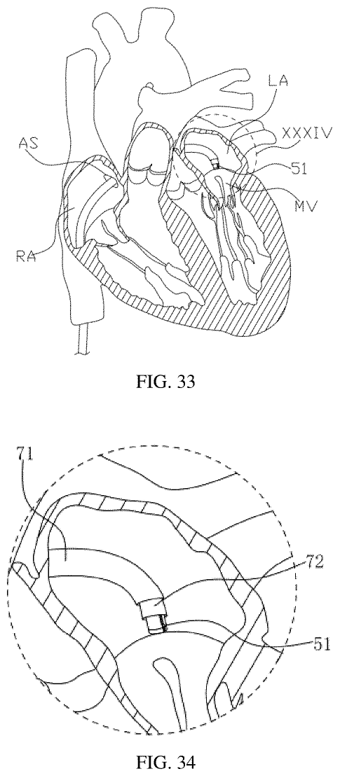

Block 312: referring to FIGS. 33-37, implanting the first anchoring component 34 into the mitral annulus.

The threading ring 347 of the first anchoring component 34 is coupled to the distal end of the tightening string 32, and the proximal end of the tightening string 32 is coupled to the distal end of the delivery member 53. The anchoring apparatus 10 is inserted into the lumen of the delivery sheath 51. Specifically, firstly, detachably coupling the first anchoring component 34 to the distal end of the anchoring apparatus 10 and inserting the first anchoring component 34 into the delivery sheath 51. At this time, the threading ring 347 of the anchoring component 34 is located outside the delivery sheath 51; the tightening string 32 and the delivery member 53 are located outside the delivery sheath 51. Secondly, pushing the delivery sheath 51 distally to the left atrium in the second guide sheath 72, and adjusting the distal end of the delivery sheath 51 to face one of the triangular areas of the anterior or posterior junction of the mitral valve and the distal end of the delivery sheath 51 abuts the mitral annulus. At this time, the proximal end of the delivery member 53 extends to outside the body. Thirdly, rotating the driving tube 12 of the anchoring apparatus 10 to drive the anchoring member 340 to rotate, so as to anchor the anchoring part into the mitral annulus. Fourthly, pulling the blocking member 55 proximally to open the opening of the through slot 512; and withdrawing the delivery sheath 51 proximally, so that the first anchoring component 34 is completely disengaged from the delivery sheath 51. Finally, decoupling the anchoring apparatus 10 from the first anchoring component 34; and withdrawing the delivery sheath 51 with the anchoring apparatus 10.

Block 313: referring to FIGS. 21 and 38, delivering the spacer 36 and the second anchoring component 34, along the delivery member 53, to the tightening string 32, and implanting the second anchoring component 34 into the mitral annulus.

Specifically, firstly, detachably coupling the second anchoring component 34 to the distal end of the anchoring apparatus 10 and inserting the second anchoring component 34 into the delivery sheath 51. Secondly, delivering the spacer 36 into the second guide sheath 72 along the delivery member 53. Thirdly, passing the proximal end of the delivery member 53 through the second anchoring component 34; and inserting the delivery sheath 51 into the second guide sheath 72. At this time, the spacer 36 is located at the distal side of the delivery sheath 51. Fourthly, pushing the delivery sheath 51 in the second guide sheath 72 so that the delivery sheath 51 pushes the spacer 36 to move until the delivery sheath 51 enters the left atrium and reaches the vicinity of the mitral annulus. At this time, the spacer 36 and the second anchoring component 34 is delivered, along the delivery member 53, to the tightening string 32, and the spacer 36 is located between the first anchoring component 34 and the second anchoring component 34. Finally, adjusting the spacing of the second anchoring component 34 and the first anchoring component 34, and driving the second anchoring component 34 to be implanted into the mitral annulus by the anchoring apparatus 10.

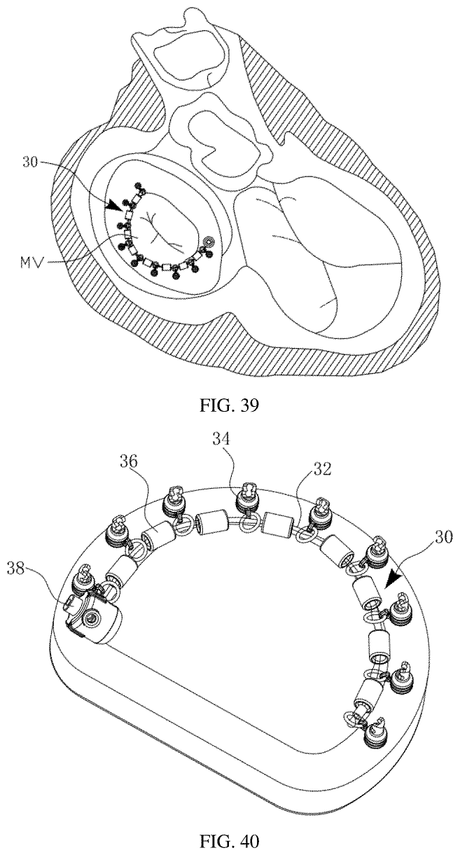

Block 314: referring to FIGS. 39 and 40, repeating Block 313 to implant the plurality of anchoring components 34 and the spacers 36 on the mitral annulus from the anterior triangular region of the mitral valve, along the posterior annulus, to the posterior triangular region or vice versa. Specifically, the anchoring components 34 and the spacers 36 are distributed on the mitral annulus. After the completion of implanting the plurality of anchoring components 34, withdrawing the second guide sheath 72, the delivery sheath 51, and the anchoring apparatus 10, and the first guide sheath 71 is retained. Of course, the first guide sheath 71 and the second guide sheath 72 can be both retained.

Block 315: delivering the retractor 38, along the delivery member 53, to the tightening string 32.

Specifically, firstly, passing the proximal end of the delivery member 53 through the retractor 38 at the distal end of the adjustment apparatus 80, and delivering the retractor 38, along the delivery member 53, to the predetermined position of the tightening string 32 in the first guide sheath 71. Secondly, rotating synchronously the rotating tube 84 and the threading rod 82 of the adjusting apparatus 80, so that the spool 383 of the retractor 38 winds the tightening string 32 three turns. Thirdly, after the delivery member 53 is withdrawn, the spool 383 continues to wind the tightening string 32 to reduce the spacing of the plurality of anchoring components 34. Finally, after a good effect of contracting the mitral annulus is achieved, stopping rotating the spool 383 so that the retractor 38 locks the tightening string 32, and the proximal end of the tightening string 32 is completely retracted into the casing 381 of the retractor 38. At this time, reversing the threading rod 82 to disengage the retractor 38 from the adjusting apparatus 80, so as to facilitate the withdrawal of the adjustment apparatus 80.

It is noticed that all operations were performed under the guidance of medical imaging device (ultrasonic equipment or digital subtraction angiography equipment or computed tomography equipment). Before the delivery member 53 is withdrawn, the coupling part between the tightening string 32 and the delivery member 53 should be observed to prevent it from being retracted into the retractor 38, which making it difficult to withdraw the delivery member 53.

It is worth noting that the above is the operation of using the transcatheter annuloplasty system 1 to implant the implant 30 into the mitral annulus to contract the mitral annulus. The method for implanting the implant 30 into tricuspid annulus, the left ventricle wall or the right ventricle wall operates similarly to the steps described above. That is to say, the transcatheter annuloplasty system 1 of the present disclosure can be configured to contract the ventricular wall during ventricular volume reductio.

In the description of this specification, reference to the terms “in some embodiments,” “in other embodiments,” “examples,” “specific examples,” or “some examples,” or the like, means specific details described in connection with the embodiments or examples. A feature, structure, material or characteristic is included in at least one embodiment or example of the present disclosure. In this specification, schematic representations of the above terms do not necessarily refer to the same embodiment or example. Furthermore, the particular features, structures, materials or characteristics described may be combined in any suitable manner in any one or more embodiments or examples.

The above are some embodiments of the present disclosure. It should be pointed out that for those of ordinary skill in the art, some improvements and modifications can be made without departing from the principles of the present disclosure, and these improvements and modifications are also regarded as the protection scope of the present disclosure.

What is claimed is:

1. A method for contracting heart tissue using a transcatheter annuloplasty system, wherein the transcatheter annuloplasty system comprises a plurality of anchoring components, a tightening string and a delivery member, the tightening string and the delivery member are both flexible, a distal end of the tightening string is coupled to a first of the plurality of anchoring component, a distal end of the delivery member is coupled to a proximal end of the tightening string, and the method comprises:
implanting the first of the plurality of anchoring component into the heart tissue, the tightening string entering a body of a patient along with the first of the plurality of anchoring component, and a proximal end of the delivery member extending outside the body;
delivering each of remaining of the plurality of anchoring components, along the delivery member, to the tightening string, and implanting each of remaining of the plurality of anchoring components into the heart tissue in sequence;
tightening the tightening string thereby adjusting a spacing of the plurality of anchoring components to contract a size of the heart tissue.

2. The method according to claim 1, wherein the method further comprises:
separating the delivery member from the tightening string, and withdrawing the delivery member.

3. The method according to claim 2, wherein the transcatheter annuloplasty system further comprises a retractor, tightening the tightening string comprises:
delivering the retractor, along the delivery member, to the tightening string;
before the delivery member is separated from the tightening string, driving the retractor to wind the tightening string to tighten the tightening string until the heart tissue contracts to a desired size.

4. The method according to claim 3, wherein when the retractor stops winding the tightening string, the tightening string is locked by the retractor.

5. The method according to claim 3, wherein the transcatheter annuloplasty system further comprises an adjusting apparatus for delivery and controlling the retractor;
driving the retractor to wind the tightening string comprises:
driving the adjusting apparatus to drive the retractor, so as to wind the tightening string.

6. The method according to claim 2, wherein the transcatheter annuloplasty system further comprises a retractor, tightening the tightening string comprises:
delivering the retractor, along the delivery member, to the tightening string;
before the delivery member is separated from the tightening string, driving the retractor being to wind the tightening string;
after the delivery member is separated from the tightening string, driving the retractor to continue winding the tightening string until the heart tissue contracts to a desired size.

7. The method according to claim 6, wherein when the retractor stops winding the tightening string, the tightening string is locked by the retractor.

8. The method according to claim 6, wherein the transcatheter annuloplasty system further comprises an adjusting apparatus for delivery and controlling the retractor;
driving the retractor to wind the tightening string comprises:
driving the adjusting apparatus to drive the retractor ao as to wind the tightening string.

9. The method according to claim 2, wherein the transcatheter annuloplasty system further comprises a locking pin;
tightening the tightening string comprises:
before the delivery member is separated from the tightening string, tightening the tightening string until the heart tissue contracts to a desired size;
delivering the locking pin, along the delivery member, to the tightening string;
driving the locking pin to lock the tightening string.

10. The method according to claim 1, wherein the transcatheter annuloplasty system further comprises at least one spacer, the method further comprises:
delivering the at least one spacer in sequence, along the delivery member, to the tightening string, and one of the at least one spacer being provided between at least two adjacent anchoring components.

11. The method according to claim 10, wherein the transcatheter annuloplasty system further comprises a delivery sheath, and the anchoring component is movably inserted therein;
delivering the at least one spacer in sequence, along the delivery member, to the tightening string, comprises:
after the proximal end of the delivery member being passed through one of the at least one spacer, pushing the spacer to be delivered along the delivery member;
abutting a distal end of the delivery sheath to the spacer;
pushing the delivery sheath to move distally, to push the spacer to continue moving, along the delivery member, until the spacer is delivered onto the tightening string.

12. The method according to claim 1, wherein the transcatheter annuloplasty system further comprises a delivery sheath and an anchoring apparatus, the anchoring component is detachably coupled to a distal end of the anchoring apparatus and inserted into the delivery sheath, the anchoring apparatus is configured to drive the anchoring component to be implanted into the heart tissue.

13. The method according to claim 12, wherein implanting the first of the plurality of anchoring component into the heart tissue comprises:
detachably coupling the first of the plurality of anchoring component to the distal end of the anchoring apparatus and inserting the first of the plurality of anchoring component into the delivery sheath;
pushing the delivery sheath until a distal end of the delivery sheath abuts the heart tissue;
driving the anchoring apparatus to drive the first of the plurality of anchoring component to be implanted into the heart tissue.

14. The method according to claim 12, wherein delivering each of remaining of the plurality of anchoring components, along the delivery member, to the tightening string and implanting each of remaining of the plurality of anchoring components into the heart tissue in sequence comprises:
detachably coupling one of the remaining of the plurality of anchor components to the distal end of the anchoring apparatus and inserting the one of the remaining of the plurality of anchor components into the delivery sheath;
coupling the anchoring component to the proximal end of the delivery member;
pushing the delivery sheath until a distal end of the delivery sheath abuts the heart tissue, and the anchoring component is delivered, along with a pushing of the delivery sheath, along the delivery member, to the tightening string;
driving the anchoring apparatus to drive the anchoring component to be implanted into the heart tissue.

15. The method according to claim 12, wherein the anchoring component comprises an anchoring member and a threading structure, the anchoring member is configured to be anchored into the heart tissue, and the threading structure is configured to couple the tightening string.

16. The method according to claim 15, wherein the anchoring member is rotated to anchor into the heart tissue, and the threading structure is rotatably sleeved on the anchoring member.

17. The method according to claim 15, wherein a tube wall of the delivery sheath defines a through slot extending from a distal end of the delivery sheath towards a proximal end of the delivery sheath, and the through slot interconnects with a lumen of the delivery sheath, a distal end of the through slot defines an opening, and the method further comprises:
after the anchoring component is detachably coupled to the distal end of the anchoring apparatus, withdrawing the anchoring apparatus proximally so that the anchoring member is received into the lumen of the delivery sheath, wherein the threading structure is at least partially located outside the delivery sheath such that the tightening string is separated from the anchoring member.

18. The method according to claim 17, wherein the transcatheter annuloplasty system further comprises a blocking member, the blocking member extends along an axial direction of the delivery sheath, a distal end of the blocking member is adapted to a circumference of the delivery sheath to close the opening of the through slot;
the method also comprises:
before the anchoring apparatus is withdrawn proximally, pulling the blocking member proximally to open the opening of the through slot;
after the anchoring apparatus is withdrawn proximally, releasing the blocking member such that the blocking member closes the opening of the through slot.

19. The method according to claim 18, wherein the method further comprises:
after the anchoring component is implanted into the heart tissue, pulling the blocking member proximally to open the opening of the through slot to release the anchoring component from the delivery sheath.

20. The method according to claim 1, wherein the transcatheter annuloplasty system further comprises a guide apparatus having a channel, the method further comprises:
before the first of the plurality of anchoring component is implanted into the heart tissue, establishing a delivery pathway from outside the body to the heart tissue through the guide apparatus.

21. The method according to claim 1, wherein the heart tissue is one of a mitral annulus, a tricuspid annulus, a left ventricular wall, and a right ventricular wall.

22. A method for contracting heart tissue using a transcatheter annuloplasty system, wherein the transcatheter annuloplasty system comprises a plurality of anchoring components, a tightening string and a delivery sheath; the tightening string is flexible, a distal end of the tightening string is coupled to a first of the plurality of anchoring component, a tube wall of the delivery sheath defines a through slot extending from a distal end of the delivery sheath towards a proximal end of the delivery sheath, and a part of the anchoring component exposed from the through slot, the method comprises:
implanting the first of the plurality of anchoring component into the heart tissue;
delivering each of remaining of the plurality of anchoring components to the tightening string and implanting each of remaining of the plurality of anchoring components into the heart tissue in sequence; and
tightening the tightening string to adjust a spacing of the anchoring components to contract a size of the heart tissue.

23. The method according to claim 22, wherein the transcatheter annuloplasty system further comprises an anchoring apparatus for driving the anchoring component to be implanted into the heart tissue; the anchoring component comprises an anchoring member and a threading structure; the anchoring member is configured to be anchored into the heart tissue; the threading structure is configured to couple the tightening string;
a distal end of the through slot defines an opening; the method also comprises:
detachably coupling the anchoring component to a distal end of the anchoring apparatus and inserting the anchoring component into the delivery sheath, so that the anchoring component is received in the lumen of the delivery sheath, and the threading structure enters the through slot and is partially outside the delivery sheath.

24. The method according to claim 23, wherein the transcatheter annuloplasty system further comprises a blocking member, the blocking member extends along an axial direction of the delivery sheath, a distal end of the blocking member is adapted to a circumference of the delivery sheath to close the opening of the through slot;
the method also comprises:
before the anchoring component is inserted into the delivery sheath, pulling the blocking member proximally to open the opening of the through slot;
after the anchoring component is inserted into the delivery sheath, releasing the blocking member so that the blocking member closes the opening of the through slot.

25. The method according to claim 24, wherein the method further comprises:
after the anchoring component being implanted into the heart tissue, pulling the blocking member proximally to open the opening of the through slot to release the anchoring component from the delivery sheath.

26. The method according to claim 22, wherein the heart tissue is one of a mitral annulus, a tricuspid annulus, a left ventricular wall, and a right ventricular wall.

27. A transcatheter annuloplasty system, wherein the transcatheter annuloplasty system comprises a plurality of anchoring components, a tightening string, a delivery sheath and a blocking member;
a tube wall of the delivery sheath defines a through slot extending from a distal end of the delivery sheath towards a proximal end of the delivery sheath; the through slot interconnects with a lumen of the delivery sheath; a distal end of the through slot defines an opening, the anchoring component is movably inserted into the delivery sheath and a part of the anchoring component exposed from the through slot; the tightening string is coupled to the anchoring component; the blocking member is movably arranged on the delivery sheath and configured to close the opening of the through slot to separate the tightening string from anchoring component.

28. The system according to claim 27, wherein the blocking member extends along an axial direction of the delivery sheath, a distal end of the blocking member is adapted to a circumference of the delivery sheath to close the opening of the through slot.

29. The system according to claim 28, wherein the distal end of the blocking member closes the opening of the through slot when no external force is applied on the blocking member.

30. The system according to claim 28, wherein at least the distal end of the blocking member is made of shape memory materials.

31. The system according to claim 28, wherein at least the distal end of the blocking member is elastic.

32. The system according to claim 28, wherein the distal end of the blocking member is a non-closed loop in a natural state.

33. The system according to claim 28, wherein the distal end of the blocking member comprises an arc-shaped segment in a natural state.

34. The system according to claim 27, wherein when the anchoring component is movably inserted into the delivery sheath, the part of the tightening string extending to the distal end from where it is coupled to the anchoring component is located outside.

35. The system according to claim 34, wherein the anchoring component comprises an anchoring member and a threading structure, the anchoring member is configured to be anchored into the heart tissue, and the threading structure is configured to couple the tightening string.

36. The system according to claim 35, wherein the anchoring member is received in the lumen of the delivery sheath; the threading structure is at least partially exposed from the through slot and is located outside the delivery sheath; the tightening string is coupled to the part of the threading structure outside the delivery sheath.

37. The system according to claim 36, wherein the threading structure comprises a connecting member and a threading ring; the connecting member is movably sleeved on the anchoring member; the threading ring is movably coupled with the connecting member; the threading ring is configured to couple the tightening string.

38. The system according to claim 37, wherein a holding end is protruded from one side of the connecting member away from the anchoring member; a radial width of the holding end is adapted with a radial width of the through slot; the holding end is located in the through slot.
